SEQUENCE LISTING (43 sequences)

SEQ ID NO: 1 - DNA, 11017 bp, Homo sapiens
  source 1..11017: mol_type = genomic DNA
ctgcgtgttttcggtccaaatccttttctttttctccctcccgtcaagatagtggtttccactccctgctctcgccaggacaccgccttttggactggggctgaattctgccccttgaagctctgctccttggagctgggggccccagcggtaggcggagttgattggagacctgccacccacattccgaccccaagcgacctccgagagggcggggtctcaggctgggttatttagctcgtccacccttctccaccagaaggagcgaaacatctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggtgagctccccagagccgggcggggtgggctgcgcccctggctccgagtcagtcaaataccggggaggacatgcgcccccgttggcctctggccgattctctttgccaagggcccgcgcattgggctcaaagcgcgcggaggccacgggggtggggggctttagggcagtcggtggcacacagtaagcgctcaataaatgttgccgctacccgatctccttggaattgagggcatgctcctgtgcgcccctcattgcacaaagctcccttgtctggaaactcagcagtcactggagccgccgtcggtcctctcgctttctctattttaaaaaccgtttcaaccgctagcgctgttggatctctacctttcaggctttatcacctggggtggagtgggtggtgagagggaagggatggggagtcaagacaggagaacgctctcgatttcccgacccacctcccgtcatatctcatgtcacatctaccgggtgttctacccggtcctgttaactttttttcttccgcgaaagcccaaattcccatcactgctctagctctctcccagatccccaaatccggatgtctgtctcagggaacgtcaaagcaacatgcccggatgaggtcctcccctctccccacctcccagccctctcctcctgcctcccccggtgtcacccgcgcctccttcctctcttcacctccctctaaacaagaatccggcgcagtcagttctagaccctgagcatctcccgcgtccctgcccaatatccgtcccgcccccgaccgcccgcgtcctcttgaacctgcggagccctgctccccgcactccacgcccttgctccgagcttccgagaaagcggggcaaagggagccaggaagagagaaaatgcgcacggagcaggtggccccgcgactccatggtgaggggacggggcacggaaggtggaaaggcgcgctccctcctcccttttttcctccctatggtgggcagttggtgacacacagtaaaggctcaataaatgttcccgctactcgatctccttggaattaaggggatgctcctgagcgcccctcattccacacagctctggtgcctggaaactgcggtcgccggagcggtcgtcggtcctcccggtttctctattttaaaagccatttcaaacctccagcggcctcgccacctctgctgcagaccttccaagcccttctgaagccggaatgatttctccctaattcactccccctgcttaacacattcgaacggctcccgcttgttctcgggaccaaatccaaagcccctggcgccaccttcgagagcctccggacctgctgaccccccacacctgggctctgagctccagctccaagatgctgctcgccctcccttccccgccgctctctttctgcgcgcctttcccctgccttccctttctgtcactctccctggctgagccctctccgcctccaggtctccgggaaggttgtccccaggccggtggggcccctgcaggtcctcagcctcgggcggggtggggtggggggtcgcacagcgggaattgccaccagcgcgtccgggctccacagcgcgctcgtccagggcgcggcagcgctcagcccagcgcttggcacagtcagtgaccacagggaggagaaagtgaaggcaggagcgcaccttcccacctgccgtccccgtcccctcagaaccgagtcctggggccacctgctcggcgcggtccctctctctctggctcagtccccgcaccccgatacgtctcctcctctcagaggctccgaggaagggcgtgggcgctggagggagcagggctcagccgggtgcccctcccgccaggccccaccggggctgagcctcttctgtcgcccgcaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggtaagcgcggggctgagggtgtcaggaggcctctgggctgtgtgagaagctggggggatgcgcgtgtggagaagagggcgcaggatgggggtgctggaacctggagcgccggggtctttgggggtgtcgaaggggcggtggctgcagctggcacaaagtggctggagcctgggggaggggcgtgatgctgaaggggcagaggccacacagaaggacggggtgctgaggctctcctggcagagacaagaagaggcgctcaccaaaagtcactggccaagtcctcctctgtccttctaggccgcactgtgggcgaggaccctcagggcctgttccagaaacaccgggaagaagagctggaagagagaaggaagttgtaccggtgagccctcctcccctgaccccacgtgagctgctgatgcttccagcacccatacttgatttccttcctgccccacaggtggggaaactggaaggacgggttaattctgaatatggctggggccaaactatatgacctccctgtggatgagcgatttctggaagacaagagagttgactttgaggtttcgctggccaaggggtgagagcaaggggaggctgggtgagagggaggtgtcctggtctagtggaagccaaggggcttatgggctgcactgcattggactggcccaggatcggtgcctgtggtcgtcatgttagagcctcagaatgatgctcaaaccctttgccccatcctgccctgaaggctggccgacctcgctatcaaagactctctaaatgttctgacttgctggaaggatctagatgacttcaaccggattttctggtgtggtcagagcaagctggctggtcagtcccccaccccagtatgtctcccaaccccccagatcccacccagatcccacccaacccaggggaattgaaagaagcagggtggggagaccagagacttgggtccctctggtgggctggagtcaaggaggcatggttggtggggttggaaggaccaagagctcagatcccacaacttgctcaacaactgccttccccagagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggaggtatggacatcagagccctgaggaagctcagcagtgaagtggggtggcctagtgccaatgatgctgctgcgggacccactgtgggcctggcttgctgccagccagcaaggacggattctgcaggagaggtcctgagggaccctggagaagctcagctgctcggcctccttcctacacgagagtagctgcggagggagggcgtgcaagatggaatggttgatagaaacaaggtcaaatgaagaaatgtgactggcccctggtgctagggattcggggagtgtgcagagtaagagagtcaggatcagagtgctgtggctgttcatcaggaggcgatatggaacaacagagaggaaattcatgggattaagggcagataggcttggatgtgaatcctggttctgctatttgtaagctgtgtgacctcaggcaaaatacccaacctctctgattatctgtttcctcttctgtaaaattgaggctcattcaactaggtataaaaattataaggctgagcacagtgactcacacctgtaatcccagcactttgggaggctgcaggaggatcacctgagcccaggagttggagaccagcctaggcaacgtggtgaaaccctgtctctacaaaagtacaaaaattatcacctgtaatctttggtgtagtggcatgcacctgtagtcccagctacttgggaggctgaggtgggaggatcacctgggcccaaggaagtcaaggctgcagtgagccgtggtgatcctgccacgcactccagcctgggcagcacggtaagaccctgattaaaaaaaaaaatgatatgaggaagtaagcacatgatccattacctggtataaagtagatgatggagacgagttagctccactctcttcccccttggaggaatccgagtaggaagaagatgctttatgttgatagcagcccttgacctcttcccccacccagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccaggtgagaggactcaggatttctgctcccagtctctggcttttgagaaagctcagccccttatcaaaatttactgagcaccatctgcaaaggcactgggctaaggcacctgggagacgcgaaaggaggaaatgcgccaagtctctattcgaaggccttgcagtgtagctggagagacagactgggccagtctgcagaagctccaggactgaggaagtccctgaggacagagacagcccaggagggattggggaggtggggtgtcacgagctttggaggagggccaggtctcagacaagcaggagggagaggaaggaccggtcctcaggggcaagacagactgggcagaggctgaggtagaggagtcaggcaattcctaggggacagttggaccagaagccagactaagccaggctaaggagtatgaggtgggaagctggagaaggccgtgatgaaacgtgctgtgggcacgcggtgtgggcagtggtgtttagggtgggttggagaggggagatgctagaaatagggactggggcaacatgccccagcactgaccccaggaggaggggagctcggatgtgggtagtgaccatcaggatggagaggacaggtggatgcgagtgcagagggagatcaaggagaggggaaaggggagccaggccagttgcctgcagaaatagcaccgataaagagaacagggtgcccaggtggatttttgtctggccaatgttgaggtaacgggaagacacttaaattatccagcaggcagctgagaacaagagattcagtcatttgtgtagaggtggggccaaaagatgagttttcaaaaaacagtgtgggatggaggaagcaggtggtttgaatcttgtccacactgtgggaactgaaaggggaagtggaggctggaggaggcaccagaagagcagtttctgcagaatgctggaggcaaagcagcttgcagggtgctaatgcactgagcgggcaggaaggggaggggaggaaacgggagccgtagggagcggcaggagtgagatggggaaagcagaacaggtttggaggacgaagggtgaggttaacatgcaaggggggcgggaatcgctgagtgcctggcactcatgccttctctccccacacttgtccctgctccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaaggtaactccttatccccttctctcttgcctgccactatctctgccccagggcacgttccgacctctggaggctccctctctgtggggtctcggggtacagagagaaacaatgaatggacaatgtgagagcaacagagaatgagcagggctggccatgcattttcaaaggggatagcacctcccacctgggaaagtggcgaaaataatcttactctttttatatgtaaagcacagtacatgcaaatgaccaaggattccatccaacaagaggaacccgtgaaataacgttaaaaagatcttttccagacattgattttttttttttttttgagatggagtctccctttgcccagactggagtgcagtgatgcgatctcggctcactgcaacctctgcctcctgggtttaagcaattctcccacttcagcctccccagtagctgggattacagtcgcgcaccaccaggcctggctaattttttttgtatttttaatagagacaggatttcaccatgttggccaggctggtctcgaatgcctgacctcagatgatctgcttgtctaggtctcccaaagtgctgggattacaggcgtgagtcactgtgcctggtccaagtttgtcttctttaaagaactgaaagaagcctagtgtaatggatgaaggagagagggaggagaccaggctctggagggaggcagtggttagaacatccattctcagtgggggcacaatgtcccccaagggatgaagatggttggaaaatgtgtgtgaaaaatattattattattattattattattgctgcccaggctggagtgcagtgtcacgatcttggctcactgcaacctctgcctcctgggttcaagcaattctcctgcttcagcctcccgagtagctgggattcaggtgcctgccaccatgcctggctaatttttgtatttttagtagagatgaggttttgccatgttggtcaggctggtctgaaactcctgacctcaggtgatccgcccacctcggcctcccaaagtgttgggattacaggcgtgagccaccgtggccggccaaatcttattactttatatagcacagatataatgtgatatatatctatataaagcacagatataatgcaatacatagacagatatacagtatatatgtagcattaatatttaatgaggtggagtgagattaggaaaaaacatctcaaaagggtacttagtgggtgattaaaaaaaaaaaagttgagaaatcttggtctagacctatgaaaaaatatgaaaagaaaaggaggccaggcacggtggttcacgcctgtaatcccagctactcgggaggctgaggcaggagaatcgcgtgaacccagaagacggaggttgcagtgagccgagatcacaccactgtacttcagcctgggaaacagagcaagactccatctcaaaacaaaacaacaacaacaaagaatgatttctttcaattatcataaagcactaagtctggtttgtggaatggaaatagttgatgcaaatcaactgaaagctcagtggttttatttttatgtatttatttttaactttaattttaggttcaaggatacatgggcaggtttgttctgtaagtaaattgtgtgtcatggggcttggtgtacagattattttgtcaccaggtaataagcatggtaccccataggtagtttttggatcccctcactccttccaccctctgtccacaagtagatgctggtgtctgttgttcccttatttgtgtccatgtgtactcaatgtttagctctcacttataagtaagaacataccatatttggctttctgttccagtgttagtgcacttaggattatggcctccagctccatccatgttgctgcagagaacatgatcttgttcttcttcatggctgcatagtattccacagcatatgtctaccacattttctttacctggtctattgttgatgggcatttaggttgattccatatctttgctattgtgagtagtgctggatgaacatccgtgagcatgtgtgtttatagtaaggcgatttatattttgggaggtatatatccagtaatgggattgctgggttgaatggtacttctgaaaactcactgttgagaggccactgaactttacgtctgtgctttttctgaagacctggtgcctttcctgctgtcagcccttgtttcctgcagggatcttgcagctctcctctgactctgcccttccctgtgtttctccctctagcttataattccccacctgcgatacaccctggaaattaacgtccgggccaggactgggctggtctctgacatgggaattttcgaccaggtatgggaagagaaagggagattctgggtctgtcttttttccagctctggattcagggcaggtcgattcgcctgaccctgctcaccctcacttctcagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtgaggcaagcgggaaggccagtgggggtgcaagtgggggtggagaagacatgtaggagagcaggaggtctgcgtctggttgggggcctggggccctgacctggccatgtgagcaggggcagagctggcttcagctccctggccctgctccgttggttggtaggtatgtggaaggaatcgtgagtctccactataagacagacgtggctgtgaaagacgacccagagctgcagacctggtgtcgagagatcactgaaatcgggctgcaaggggcccaggaccgaggtaagaggagcccctgccctgagatctcagacacaaagcccaagagatcttcccagaatcccctgtgcttctgtgaaatctcccagaagcattttcaacacctatgagaactccagaggccttctcagattccactccctgtcacctagagacaggtccccgtcctacacactgagaacctctaggtgccagatgcagcgggaccagtggctgctcataaatgtttaacaactgactctcgggaagaaccgtcctgatttgtagcttttgcacatttccatggtataaatatttttactgtgactaccaaggtgatgctgaccagcttgctaaacacctaacgtcatggactgactcttgcgagccagtgtgagtcagcagcagcaccccactgagtgggaccctcccggcagagctccccactcccccaccccaacctgctgtcatggtaattcctgtaagagttaactgagcattcctgccgctctttctcgcccttgttctcctctcttggcctcttcctctgagcgctgccctactcctgccttcgcagcttctaactctcttcaccccttgcagggtttcctgtctctttacaggctcgggaccaggtttgccactttgtcaccatgtgtatcttcacctgcaccggccaacacgcctctgtgcacctgggccaggtacttaccagagatgggcagctgggaatttggggcctggagtgagggaggggtctgtgtgcaggtggaccacggggctcgcgtgcttggtaggcactgactctggatccctgcccttccttagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtgagagctgagtgcccagggccctaaggaaggaggcagctgtggggacgtgtggcatcccagactgggggtcataaggctctcagccaccttttcctctccctcccaggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaa

SEQ ID NO: 2 - DNA, 11017 bp, Homo sapiens
  source 1..11017: mol_type = genomic DNA
ctgcgtgttttcggtccaaatccttttctttttctccctcccgtcaagatagtggtttccactccctgctctcgccaggacaccgccttttggactggggctgaattctgccccttgaagctctgctccttggagctgggggccccagcggtaggcggagttgattggagacctgccacccacattccgaccccaagcgacctccgagagggcggggtctcaggctgggttatttagctcgtccacccttctccaccagaaggagcgaaacatctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggtgagctccccagagccgggcggggtgggctgcgcccctggctccgagtcagtcaaataccggggaggacatgcgcccccgttggcctctggccgattctctttgccaagggcccgcgcattgggctcaaagcgcgcggaggccacgggggtggggggctttagggcagtcggtggcacacagtaagcgctcaataaatgttgccgctacccgatctccttggaattgagggcatgctcctgtgcgcccctcattgcacaaagctcccttgtctggaaactcagcagtcactggagccgccgtcggtcctctcgctttctctattttaaaaaccgtttcaaccgctagcgctgttggatctctacctttcaggctttatcacctggggtggagtgggtggtgagagggaagggatggggagtcaagacaggagaacgctctcgatttcccgacccacctcccgtcatatctcatgtcacatctaccgggtgttctacccggtcctgttaactttttttcttccgcgaaagcccaaattcccatcactgctctagctctctcccagatccccaaatccggatgtctgtctcagggaacgtcaaagcaacatgcccggatgaggtcctcccctctccccacctcccagccctctcctcctgcctcccccggtgtcacccgcgcctccttcctctcttcacctccctctaaacaagaatccggcgcagtcagttctagaccctgagcatctcccgcgtccctgcccaatatccgtcccgcccccgaccgcccgcgtcctcttgaacctgcggagccctgctccccgcactccacgcccttgctccgagcttccgagaaagcggggcaaagggagccaggaagagagaaaatgcgcacggagcaggtggccccgcgactccatggtgaggggacggggcacggaaggtggaaaggcgcgctccctcctcccttttttcctccctatggtgggcagttggtgacacacagtaaaggctcaataaatgttcccgctactcgatctccttggaattaaggggatgctcctgagcgcccctcattccacacagctctggtgcctggaaactgcggtcgccggagcggtcgtcggtcctcccggtttctctattttaaaagccatttcaaacctccagcggcctcgccacctctgctgcagaccttccaagcccttctgaagccggaatgatttctccctaattcactccccctgcttaacacattcgaacggctcccgcttgttctcgggaccaaatccaaagcccctggcgccaccttcgagagcctccggacctgctgaccccccacacctgggctctgagctccagctccaagatgctgctcgccctcccttccccgccgctctctttctgcgcgcctttcccctgccttccctttctgtcactctccctggctgagccctctccgcctccaggtctccgggaaggttgtccccaggccggtggggcccctgcaggtcctcagcctcgggcggggtggggtggggggtcgcacagcgggaattgccaccagcgcgtccgggctccacagcgcgctcgtccagggcgcggcagcgctcagcccagcgcttggcacagtcagtgaccacagggaggagaaagtgaaggcaggagcgcaccttcccacctgccgtccccgtcccctcagaaccgagtcctggggccacctgctcggcgcggtccctctctctctggctcagtccccgcaccccgatacgtctcctcctctcagaggctccgaggaagggcgtgggcgctggagggagcagggctcagccgggtgcccctcccgccaggccccaccggggctgagcctcttctgtcgcccgcaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggtaagcgcggggctgagggtgtcaggaggcctctgggctgtgtgagaagctggggggatgcgcgtgtggagaagagggcgcaggatgggggtgctggaacctggagcgccggggtctttgggggtgtcgaaggggcggtggctgcagctggcacaaagtggctggagcctgggggaggggcgtgatgctgaaggggcagaggccacacagaaggacggggtgctgaggctctcctggcagagacaagaagaggcgctcaccaaaagtcactggccaagtcctcctctgtccttctaggccgcactgtgggcgaggaccctcagggcctgttccagaaacaccgggaagaagagctggaagagagaaggaagttgtaccggtgagccctcctcccctgaccccacgtgagctgctgatgcttccagcacccatacttgatttccttcctgccccacaggtggggaaactggaaggacgggttaattctgaatatggctggggccaaactatatgacctccctgtggatgagcgatttctggaagacaagagagttgactttgaggtttcgctggccaaggggtgagagcaaggggaggctgggtgagagggaggtgtcctggtctagtggaagccaaggggcttatgggctgcactgcattggactggcccaggatcggtgcctgtggtcgtcatgttagagcctcagaatgatgctcaaaccctttgccccatcctgccctgaaggctggccgacctcgctatcaaagactctctaaatgttctgacttgctggaaggatctagatgacttcaaccggattttctggtgtggtcagagcaagctggctggtcagtcccccaccccagtatgtctcccaaccccccagatcccacccagatcccacccaacccaggggaattgaaagaagcagggtggggagaccagagacttgggtccctctggtgggctggagtcaaggaggcatggttggtggggttggaaggaccaagagctcagatcccacaacttgctcaacaactgccttccccagagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggaggtatggacatcagagccctgaggaagctcagcagtgaagtggggtggcctagtgccaatgatgctgctgcgggacccactgtgggcctggcttgctgccagccagcaaggacggattctgcaggagaggtcctgagggaccctggagaagctcagctgctcggcctccttcctacacgagagtagctgcggagggagggcgtgcaagatggaatggttgatagaaacaaggtcaaatgaagaaatgtgactggcccctggtgctagggattcggggagtgtgcagagtaagagagtcaggatcagagtgctgtggctgttcatcaggaggcgatatggaacaacagagaggaaattcatgggattaagggcagataggcttggatgtgaatcctggttctgctatttgtaagctgtgtgacctcaggcaaaatacccaacctctctgattatctgtttcctcttctgtaaaattgaggctcattcaactaggtataaaaattataaggctgagcacagtgactcacacctgtaatcccagcactttgggaggctgcaggaggatcacctgagcccaggagttggagaccagcctaggcaacgtggtgaaaccctgtctctacaaaagtacaaaaattatcacctgtaatctttggtgtagtggcatgcacctgtagtcccagctacttgggaggctgaggtgggaggatcacctgggcccaaggaagtcaaggctgcagtgagccgtggtgatcctgccacgcactccagcctgggcagcacggtaagaccctgattaaaaaaaaaaatgatatgaggaagtaagcacatgatccattacctggtataaagtagatgatggagacgagttagctccactctcttcccccttggaggaatccgagtaggaagaagatgctttatgttgatagcagcccttgacctcttcccccacccagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccaggtgagaggactcaggatttctgctcccagtctctggcttttgagaaagctcagccccttatcaaaatttactgagcaccatctgcaaaggcactgggctaaggcacctgggagacgcgaaaggaggaaatgcgccaagtctctattcgaaggccttgcagtgtagctggagagacagactgggccagtctgcagaagctccaggactgaggaagtccctgaggacagagacagcccaggagggattggggaggtggggtgtcacgagctttggaggagggccaggtctcagacaagcaggagggagaggaaggaccggtcctcaggggcaagacagactgggcagaggctgaggtagaggagtcaggcaattcctaggggacagttggaccagaagccagactaagccaggctaaggagtatgaggtgggaagctggagaaggccgtgatgaaacgtgctgtgggcacgcggtgtgggcagtggtgtttagggtgggttggagaggggagatgctagaaatagggactggggcaacatgccccagcactgaccccaggaggaggggagctcggatgtgggtagtgaccatcaggatggagaggacaggtggatgcgagtgcagagggagatcaaggagaggggaaaggggagccaggccagttgcctgcagaaatagcaccgataaagagaacagggtgcccaggtggatttttgtctggccaatgttgaggtaacgggaagacacttaaattatccagcaggcagctgagaacaagagattcagtcatttgtgtagaggtggggccaaaagatgagttttcaaaaaacagtgtgggatggaggaagcaggtggtttgaatcttgtccacactgtgggaactgaaaggggaagtggaggctggaggaggcaccagaagagcagtttctgcagaatgctggaggcaaagcagcttgcagggtgctaatgcactgagcgggcaggaaggggaggggaggaaacgggagccgtagggagcggcaggagtgagatggggaaagcagaacaggtttggaggacgaagggtgaggttaacatgcaaggggggcgggaatcgctgagtgcctggcactcatgccttctctccccacacttgtccctgctccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaaggtaactccttatccccttctctcttgcctgccactatctctgccccagggcacgttccgacctctggaggctccctctctgtggggtctcggggtacagagagaaacaatgaatggacaatgtgagagcaacagagaatgagcagggctggccatgcattttcaaaggggatagcacctcccacctgggaaagtggcgaaaataatcttactctttttatatgtaaagcacagtacatgcaaatgaccaaggattccatccaacaagaggaacccgtgaaataacgttaaaaagatcttttccagacattgattttttttttttttttgagatggagtctccctttgcccagactggagtgcagtgatgcgatctcggctcactgcaacctctgcctcctgggtttaagcaattctcccacttcagcctccccagtagctgggattacagtcgcgcaccaccaggcctggctaattttttttgtatttttaatagagacaggatttcaccatgttggccaggctggtctcgaatgcctgacctcagatgatctgcttgtctaggtctcccaaagtgctgggattacaggcgtgagtcactgtgcctggtccaagtttgtcttctttaaagaactgaaagaagcctagtgtaatggatgaaggagagagggaggagaccaggctctggagggaggcagtggttagaacatccattctcagtgggggcacaatgtcccccaagggatgaagatggttggaaaatgtgtgtgaaaaatattattattattattattattattgctgcccaggctggagtgcagtgtcacgatcttggctcactgcaacctctgcctcctgggttcaagcaattctcctgcttcagcctcccgagtagctgggattcaggtgcctgccaccatgcctggctaatttttgtatttttagtagagatgaggttttgccatgttggtcaggctggtctgaaactcctgacctcaggtgatccgcccacctcggcctcccaaagtgttgggattacaggcgtgagccaccgtggccggccaaatcttattactttatatagcacagatataatgtgatatatatctatataaagcacagatataatgcaatacatagacagatatacagtatatatgtagcattaatatttaatgaggtggagtgagattaggaaaaaacatctcaaaagggtacttagtgggtgattaaaaaaaaaaaagttgagaaatcttggtctagacctatgaaaaaatatgaaaagaaaaggaggccaggcacggtggttcacgcctgtaatcccagctactcgggaggctgaggcaggagaatcgcgtgaacccagaagacggaggttgcagtgagccgagatcacaccactgtacttcagcctgggaaacagagcaagactccatctcaaaacaaaacaacaacaacaaagaatgatttctttcaattatcataaagcactaagtctggtttgtggaatggaaatagttgatgcaaatcaactgaaagctcagtggttttatttttatgtatttatttttaactttaattttaggttcaaggatacatgggcaggtttgttctgtaagtaaattgtgtgtcatggggcttggtgtacagattattttgtcaccaggtaataagcatggtaccccataggtagtttttggatcccctcactccttccaccctctgtccacaagtagatgctggtgtctgttgttcccttatttgtgtccatgtgtactcaatgtttagctctcacttataagtaagaacataccatatttggctttctgttccagtgttagtgcacttaggattatggcctccagctccatccatgttgctgcagagaacatgatcttgttcttcttcatggctgcatagtattccacagcatatgtctaccacattttctttacctggtctattgttgatgggcatttaggttgattccatatctttgctattgtgagtagtgctggatgaacatccgtgagcatgtgtgtttatagtaaggcgatttatattttgggaggtatatatccagtaatgggattgctgggttgaatggtacttctgaaaactcactgttgagaggccactgaactttacgtctgtgctttttctgaagacctggtgcctttcctgctgtcagcccttgtttcctgcagggatcttgcagctctcctctgactctgcccttccctgtgtttctccctctagcttataattccccacctgcgatacaccctggaaattaacgtccgggccaggactgggctggtctctgacatgggaattttcgaccaggtatgggaagagaaagggagattctgggtctgtcttttttccagctctggattcagggcaggtcgattcgcctgaccctgctcaccctcacttctcagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtgaggcaagcgggaaggccagtgggggtgcaagtgggggtggagaagacatgtaggagagcaggaggtctgcgtctggttgggggcctggggccctgacctggccatgtgagcaggggcagagctggcttcagctccctggccctgctccgttggttggtaggtatgtggaaggaatcgtgagtctccactataagacagacgtggctgtgaaagacgacccagagctgcagacctggtgtcgagagatcactgaaatcgggctgcaaggggcccaggaccgaggtaagaggagcccctgccctgagatctcagacacaaagcccaagagatcttcccagaatcccctgtgcttctgtgaaatctcccagaagcattttcaacacctatgagaactccagaggccttctcagattccactccctgtcacctagagacaggtccccgtcctacacactgagaacctctaggtgccagatgcagcgggaccagtggctgctcataaatgtttaacaactgactctcgggaagaaccgtcctgatttgtagcttttgcacatttccatggtataaatatttttactgtgactaccaaggtgatgctgaccagcttgctaaacacctaacgtcatggactgactcttgcgagccagtgtgagtcagcagcagcaccccactgagtgggaccctcccggcagagctccccactcccccaccccaacctgctgtcatggtaattcctgtaagagttaactgagcattcctgccgctctttctcgcccttgttctcctctcttggcctcttcctctgagcgctgccctactcctgccttcgcagcttctaactctcttcaccccttgcagggtttcctgtctctttacaggctcgggaccaggtttgccactttgtcaccatgtgtatcttcacctgcaccggccaacacgcctctgtgcacctgggccaggtacttaccagagatgggcagctgggaatttggggcctggagtgagggaggggtctgtgtgcaggtggaccacggggctcgcgtgcttggtaggcactgactctggatccctgcccttccttagctggactggtactcttgggtgcctaatgcaccctgcatgatgcggctgcccccgccaaccaccaaggatgcaacgctggagacagtgatggcgacactgcccaacttccaccaggcttctctccagatgtccatcacttggcagctgggcagacgccagcccgttatggtgagagctgagtgcccagggccctaaggaaggaggcagctgtggggacgtgtggcatcccagactgggggtcataaggctctcagccaccttttcctctccctcccaggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaa

SEQ ID NO: 3 - RNA, 2707 nt, Homo sapiens
  source 1..2707: mol_type = mRNA
catctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaaaaaaaaaaaaaaaaaaaaaaaaa

SEQ ID NO: 4 - RNA, 2707 nt, Homo sapiens
  source 1..2707: mol_type = mRNA
catctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcatgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaaaaaaaaaaaaaaaaaaaaaaaaa

SEQ ID NO: 5 - DNA, 2707 bp, Homo sapiens
  source 1..2707: mol_type = other DNA
catctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcacgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaaaaaaaaaaaaaaaaaaaaaaaaa

SEQ ID NO: 6 - DNA, 2707 bp, Homo sapiens
  source 1..2707: mol_type = other DNA
catctttgagcaagatgggtctctaccgcatccgcgtgtccactggggcctcgctctatgccggttccaacaaccaggtgcagctgtggctggtcggccagcacggggaggcggcgctcgggaagcgactgtggcccgcacggggcaaggagacagaactcaaggtggaagtaccggagtatctggggccgctgctgtttgtgaaactgcgcaaacggcacctccttaaggacgacgcctggttctgcaactggatctctgtgcagggccccggagccggggacgaggtcaggttcccttgttaccgctgggtggagggcaacggcgtcctgagcctgcctgaaggcaccggccgcactgtgggcgaggaccctcagggcctgttccagaaacaccgggaagaagagctggaagagagaaggaagttgtaccggtggggaaactggaaggacgggttaattctgaatatggctggggccaaactatatgacctccctgtggatgagcgatttctggaagacaagagagttgactttgaggtttcgctggccaaggggctggccgacctcgctatcaaagactctctaaatgttctgacttgctggaaggatctagatgacttcaaccggattttctggtgtggtcagagcaagctggctgagcgcgtgcgggactcctggaaggaagatgccttatttgggtaccagtttcttaatggcgccaaccccgtggtgctgaggcgctctgctcaccttcctgctcgcctagtgttccctccaggcatggaggaactgcaggcccagctggagaaggagctggagggaggcacactgttcgaagctgacttctccctgctggatgggatcaaggccaacgtcattctctgtagccagcagcacctggctgcccctctagtcatgctgaaattgcagcctgatgggaaactcttgcccatggtcatccagctccagctgccccgcacaggatccccaccacctccccttttcttgcctacggatcccccaatggcctggcttctggccaaatgctgggtgcgcagctctgacttccagctccatgagctgcagtctcatcttctgaggggacacttgatggctgaggtcattgttgtggccaccatgaggtgcctgccgtcgatacatcctatcttcaagcttataattccccacctgcgatacaccctggaaattaacgtccgggccaggactgggctggtctctgacatgggaattttcgaccagataatgagcactggtgggggaggccacgtgcagctgctcaagcaagctggagccttcctaacctacagctccttctgtccccctgatgacttggccgaccgggggctcctgggagtgaagtcttccttctatgcccaagatgcgctgcggctctgggaaatcatctatcggtatgtggaaggaatcgtgagtctccactataagacagacgtggctgtgaaagacgacccagagctgcagacctggtgtcgagagatcactgaaatcgggctgcaaggggcccaggaccgagggtttcctgtctctttacaggctcgggaccaggtttgccactttgtcaccatgtgtatcttcacctgcaccggccaacacgcctctgtgcacctgggccagctggactggtactcttgggtgcctaatgcaccctgcatgatgcggctgcccccgccaaccaccaaggatgcaacgctggagacagtgatggcgacactgcccaacttccaccaggcttctctccagatgtccatcacttggcagctgggcagacgccagcccgttatggtggctgtgggccagcatgaggaggagtatttttcgggccctgagcctaaggctgtgctgaagaagttcagggaggagctggctgccctggataaggaaattgagatccggaatgcaaagctggacatgccctacgagtacctgcggcccagcgtggtggaaaacagtgtggccatctaagcgtcgccaccctttggttatttcagcccccatcacccaagccacaagctgaccccttcgtggttatagccctgccctcccaagtcccaccctcttcccatgtcccaccctccctagaggggcaccttttcatggtctctgcacccagtgaacacattttactctagaggcatcacctgggaccttactcctctttccttccttcctcctttcctatcttccttcctctctctcttcctctttcttcattcagatctatatggcaaatagccacaattatataaatcatttcaagactagaatagggggatataatacatattactccacaccttttatgaatcaaatatgatttttttgttgttgttaagacagagtctcactttgacacccaggctggagtgcagtggtgccatcaccacggctcactgcagcctcagcgtcctgggctcaaatgatcctcccacctcagcctcctgagtagctgggactacaggctcatgccatcatgcccagctaatatttttttattttcgtggagacggggcctcactatgttgcctaggctggaaataggattttgaacccaaattgagtttaacaataataaaaagttgttttacgctaaagatggaaaagaactaggactgaactattttaaataaaatattggcaaaagaaaaaaaaaaaaaaaaaaaaaaaaa

SEQ ID NO: 7 - protein, 662 aa, Homo sapiens
MGLYRIRVSTGASLYAGSNNQVQLWLVGQHGEAALGKRLWPARGKETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVIVVATMRCLPSIHPIFKLIIPHLRYTLEINVRARTGLVSDMGIFDQIMSTGGGGHVQLLKQAGAFLTYSSFCPPDDLADRGLLGVKSSFYAQDALRLWEIIYRYVEGIVSLHYKTDVAVKDDPELQTWCREITEIGLQGAQDRGFPVSLQARDQVCHFVTMCIFTCTGQHASVHLGQLDWYSWVPNAPCTMRLPPPTTKDATLETVMATLPNFHQASLQMSITWQLGRRQPVMVAVGQHEEEYFSGPEPKAVLKKFREELAALDKEIEIRNAKLDMPYEYLRPSVVENSVAI

SEQ ID NO: 8 - protein, 662 aa, Homo sapiens
MGLYRIRVSTGASLYAGSNNQVQLWLVGQHGEAALGKRLWPARGKETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVIVVATMRCLPSIHPIFKLIIPHLRYTLEINVRARTGLVSDMGIFDQIMSTGGGGHVQLLKQAGAFLTYSSFCPPDDLADRGLLGVKSSFYAQDALRLWEIIYRYVEGIVSLHYKTDVAVKDDPELQTWCREITEIGLQGAQDRGFPVSLQARDQVCHFVTMCIFTCTGQHASVHLGQLDWYSWVPNAPCMMRLPPPTTKDATLETVMATLPNFHQASLQMSITWQLGRRQPVMVAVGQHEEEYFSGPEPKAVLKKFREELAALDKEIEIRNAKLDMPYEYLRPSVVENSVAI

SEQ ID NO: 9 - DNA, 59 bp, synthetic construct
  misc_feature 1..59: note = ALOX15 shRNA
  source 1..59: mol_type = other DNA
ccgggaaactggaaggacgggttaactcgagttaacccgtccttccagtttcttttttg

SEQ ID NO: 10 - DNA, 58 bp, synthetic construct
  misc_feature 1..58: note = ALOX15 shRNA
  source 1..58: mol_type = other DNA
ccgggctatcaaagactctctaaatctcgagatttagagagtctttgatagctttttg

SEQ ID NO: 11 - DNA, 58 bp, synthetic construct
  misc_feature 1..58: note = ALOX15 shRNA
  source 1..58: mol_type = other DNA
ccggtgggaaatcatctatcggtatctcgagataccgatagatgatttcccatttttg

SEQ ID NO: 12 - DNA, 58 bp, synthetic construct
  misc_feature 1..58: note = ALOX15 shRNA
  source 1..58: mol_type = other DNA
ccggcctggaaggaagatgccttatctcgagataaggcatcttccttccaggtttttg

SEQ ID NO: 13 - DNA, 58 bp, synthetic construct
  misc_feature 1..58: note = ALOX15 shRNA
  source 1..58: mol_type = other DNA
ccggccagtttcttaatggcgccaactcgagttggcgccattaagaaactggtttttg

SEQ ID NO: 14 - DNA, 58 bp, synthetic construct
  misc_feature 1..58: note = ALOX15 shRNA
  source 1..58: mol_type = other DNA
ccgggccgtcgatacatcctatcttctcgagaagataggatgtatcgacggctttttg

SEQ ID NO: 15 - DNA, 59 bp, synthetic construct
  misc_feature 1..59: note = ALOX15 shRNA
  source 1..59: mol_type = other DNA
ccggtagatgacttcaaccggatttctcgagaaatccggttgaagtcatctattttttg

SEQ ID NO: 16 - DNA, 59 bp, synthetic construct
  misc_feature 1..59: note = ALOX15 shRNA
  source 1..59: mol_type = other DNA
ccggtggtactcttgggtgcctaatctcgagattaggcacccaagagtaccattttttg

SEQ ID NO: 17 - protein, 170 aa, synthetic construct
  REGION 1..170: note = PP1 inhibitory peptide
MEPDNSPRKIQFTVPLLEPHLDPEAAEQIRRRRPTPATLVLTSDQSSPEIDEDRIPNSLLKSTLSMSPRQRKKMTRTTPTMKELQTMVHHLGQQKQGEEPEGATESTGNQESCPPGIPDTGSASRPDTPGTAQKSAESNPKTQEQCGVEPRTEDSSAHMLPLDSQGASLV

SEQ ID NO: 18 - protein, 205 aa, synthetic construct
  REGION 1..205: note = PP1 inhibitory peptide
MAASTASHRPIKGILKNKTSSTSSRVASAEQPRGSVDEELSKKSQKWDEMNILATYHPADKDYGLMKIDEPSTPYHSMIGDDDDAYSDTETTEAMTPDTLAKKLAAAEGSEPKYRIREQESSGEEDSDLSPEEREKKRQFEMKRKLHYNEGLNIKLARQLISKDLHDDEEDEEMSETADGESMNTEESNQGSTPSDQRQNKSQSS

SEQ ID NO: 19 - DNA, 23 bp, synthetic construct
  misc_feature 1..23: note = ALOX15 gRNA recognition sequence
  source 1..23: mol_type = other DNA
taccaagcacgcgagccccgtgg

SEQ ID NO: 20 - DNA, 23 bp, synthetic construct
  misc_feature 1..23: note = ALOX15 gRNA recognition sequence
  source 1..23: mol_type = other DNA
gaccacggggctcgcgtgcttgg

SEQ ID NO: 21 - DNA, 23 bp, synthetic construct
  misc_feature 1..23: note = ALOX15 gRNA recognition sequence
  source 1..23: mol_type = other DNA
acggggctcgcgtgcttggtagg

SEQ ID NO: 22 - DNA, 23 bp, synthetic construct
  misc_feature 1..23: note = ALOX15 gRNA recognition sequence
  source 1..23: mol_type = other DNA
gcatccttggtggttggcggggg

SEQ ID NO: 23 - DNA, 23 bp, synthetic construct
  misc_feature 1..23: note = ALOX15 gRNA recognition sequence
  source 1..23: mol_type = other DNA
cgggggcagccgcatcgtgcagg

SEQ ID NO: 24 - DNA, 23 bp, synthetic construct
  misc_feature 1..23: note = ALOX15 gRNA recognition sequence
  source 1..23: mol_type = other DNA
ttagctggactggtactcttggg

SEQ ID NO: 25 - DNA, 23 bp, synthetic construct
  misc_feature 1..23: note = ALOX15 gRNA recognition sequence
  source 1..23: mol_type = other DNA
gggggcagccgcatcgtgcaggg

SEQ ID NO: 26 - DNA, 23 bp, synthetic construct
  misc_feature 1..23: note = ALOX15 gRNA recognition sequence
  source 1..23: mol_type = other DNA
cttagctggactggtactcttgg

SEQ ID NO: 27 - DNA, 23 bp, synthetic construct
  misc_feature 1..23: note = ALOX15 gRNA recognition sequence
  source 1..23: mol_type = other DNA
tgcatccttggtggttggcgggg

SEQ ID NO: 28 - DNA, 23 bp, synthetic construct
  misc_feature 1..23: note = ALOX15 gRNA recognition sequence
  source 1..23: mol_type = other DNA
caagagtaccagtccagctaagg

SEQ ID NO: 29 - DNA, 23 bp, synthetic construct
  misc_feature 1..23: note = ALOX15 gRNA recognition sequence
  source 1..23: mol_type = other DNA
gatgtccatcacttggcagctgg

SEQ ID NO: 30 - DNA, 23 bp, synthetic construct
  misc_feature 1..23: note = ALOX15 gRNA recognition sequence
  source 1..23: mol_type = other DNA
gtaccagtccagctaaggaaggg

SEQ ID NO: 31 - DNA, 23 bp, synthetic construct
  misc_feature 1..23: note = ALOX15 gRNA recognition sequence
  source 1..23: mol_type = other DNA
ttgcatccttggtggttggcggg

SEQ ID NO: 32 - DNA, 23 bp, synthetic construct
  misc_feature 1..23: note = ALOX15 gRNA recognition sequence
  source 1..23: mol_type = other DNA
ctgtgtgcaggtggaccacgggg

SEQ ID NO: 33 - DNA, 23 bp, synthetic construct
  misc_feature 1..23: note = ALOX15 gRNA recognition sequence
  source 1..23: mol_type = other DNA
tctgtgtgcaggtggaccacggg

SEQ ID NO: 34 - DNA, 23 bp, synthetic construct
  misc_feature 1..23: note = ALOX15 gRNA recognition sequence
  source 1..23: mol_type = other DNA
tgcttggtaggcactgactctgg

SEQ ID NO: 35 - DNA, 23 bp, synthetic construct
  misc_feature 1..23: note = ALOX15 gRNA recognition sequence
  source 1..23: mol_type = other DNA
agtaccagtccagctaaggaagg

SEQ ID NO: 36 - DNA, 23 bp, synthetic construct
  misc_feature 1..23: note = ALOX15 gRNA recognition sequence
  source 1..23: mol_type = other DNA
ctgcccttccttagctggactgg

SEQ ID NO: 37 - DNA, 23 bp, synthetic construct
  misc_feature 1..23: note = ALOX15 gRNA recognition sequence
  source 1..23: mol_type = other DNA
gacactgcccaacttccaccagg

SEQ ID NO: 38 - DNA, 23 bp, synthetic construct
  misc_feature 1..23: note = ALOX15 gRNA recognition sequence
  source 1..23: mol_type = other DNA
agagaagcctggtggaagttggg

SEQ ID NO: 39 - DNA, 23 bp, synthetic construct
  misc_feature 1..23: note = ALOX15 gRNA recognition sequence
  source 1..23: mol_type = other DNA
gatccctgcccttccttagctgg

SEQ ID NO: 40 - DNA, 23 bp, synthetic construct
  misc_feature 1..23: note = ALOX15 gRNA recognition sequence
  source 1..23: mol_type = other DNA
gtctgtgtgcaggtggaccacgg

SEQ ID NO: 41 - DNA, 23 bp, synthetic construct
  misc_feature 1..23: note = ALOX15 gRNA recognition sequence
  source 1..23: mol_type = other DNA
cagtccagctaaggaagggcagg

SEQ ID NO: 42 - DNA, 23 bp, synthetic construct
  misc_feature 1..23: note = ALOX15 gRNA recognition sequence
  source 1..23: mol_type = other DNA
gctgccaagtgatggacatctgg

SEQ ID NO: 43 - DNA, 23 bp, synthetic construct
  misc_feature 1..23: note = ALOX15 gRNA recognition sequence
  source 1..23: mol_type = other DNA
tctgcccagctgccaagtgatgg

(19) United States
(12) Patent Application Publication
(10) Pub. No.: US 2026/0097102 A1
(43) Pub. Date: Apr. 9, 2026

(54) Treatment Of Respiratory Disorders With Arachidonate 15-Lipoxygenase (ALOX15) Inhibitors

(71) Applicant: Regeneron Pharmaceuticals, Inc., Tarrytown, NY (US)

(72) Inventors: Julie Horowitz, Tarrytown, NY (US); Aris Baras, Tarrytown, NY (US)

(21) Appl. No.: 19/364,245

(22) Filed: Oct. 21, 2025

Related U.S. Application Data
(62) Division of application No. 16/725,309, filed on Dec. 23, 2019, now Pat. No. 12,472,241.
(60) Provisional application No. 62/785,899, filed on Dec. 28, 2018.

(51) Int. Cl. A61K 38/46; A61P 11/02; C12N 15/113; G01N 33/68

(52) U.S. Cl. CPC A61K 38/465; A61P 11/02; C12N 15/1137; G01N 33/6815; G01N 33/6818

(57) ABSTRACT

The present disclosure provides methods of treating patients having a respiratory disorder, methods of identifying subjects having an increased risk of developing a respiratory disorder, and methods of detecting human Arachidonate 15-Lipoxygenase (ALOX15) variant nucleic acid molecules and variant polypeptides.

### REFERENCE TO SEQUENCE LISTING

This application includes a Sequence Listing submitted electronically as an XML file named 381204489SEQ, created on Sep. 22, 2025, with a size of 87,102 bytes. The Sequence Listing is incorporated herein by reference.

### FIELD

The present disclosure relates generally to the treatment of patients having a respiratory disorder with an Arachidonate 15-Lipoxygenase (ALOX15) inhibitor, methods of identifying subjects having an increased risk of developing a respiratory disorder, and methods of detecting ALOX15 variant nucleic acid molecules and variant polypeptides.

### BACKGROUND

Asthma can result from aeroallergen-induced inflammation driven by T-helper type 2 (Th2) processes and mediated by cytokines including interleukin (IL)-4, IL-5 and IL-13. IL-13 is a pleiotropic Th2 cytokine produced by activated T cells, basophils, eosinophils, and mast cells, and it has been strongly implicated in the pathogenesis of asthma in preclinical models. Elevated levels of IL-13 have been detected in the airways in a subset of human asthma patients. While asthma is often characterized by eosinophilic infiltration of the airways, there is increasing evidence that there are other subtypes of the disease driven by alternative forms of inflammation. For example, studies of the cellular components of airway inflammation in asthma provide evidence for distinct eosinophilic and non-eosinophilic phenotypes of asthma. The identification of and development of biomarkers for asthma would be helpful.

Nasal polyps are often soft, painless, non-cancerous growths on the lining of the nasal passages or sinuses. Nasal polyps can result from chronic inflammation due to asthma, recurring infection, allergies, drug sensitivity, or some immune disorders. Larger growths or groups of nasal polyps can block nasal passages and possibly lead to breathing problems, a lost sense of smell, and frequent infections. Medications can often shrink or eliminate nasal polyps, but surgery is sometimes needed to remove them. Even after successful treatment, nasal polyps often return.

Allergic rhinitis typically causes symptoms in the nose, throat, eyes, ears, skin and/or roof of the mouth. Seasonal allergic rhinitis (e.g., hay fever) is most often caused by pollen carried in the air during different times of the year in different parts of the country. Allergic rhinitis, such as perennial allergic rhinitis, can also be caused by indoor allergens such as dried skin flakes, urine and saliva located on pet dander, mold, products from dust mites, and cockroach particles, with symptoms often occurring year-round. In addition to allergen triggers, symptoms may also result from irritants such as smoke and strong odors, or to changes in the temperature and humidity of the air.

Aspirin-exacerbated respiratory disease (AERD) is characterized by mucosal swelling of the sinuses and nasal membranes, nasal polyps, and asthma coupled with respiratory reactions to aspirin/NSAIDs. AERD is an acquired disease with average onset at 30 years of age. About 50% of patients develop AERD following a respiratory virus. AERD can cause severe asthma and remodeling of airways, and is often diagnosed by a history of respiratory reactions to COX1 inhibitors. The prevalence is estimated at 7.2% in general asthmatics (1.3 million in U.S.), 14.9% in severe asthmatics, 9.7% among patients with nasal polyps, 8.7% among patients with chronic sinusitis. However, about 20-40% of patients with nasal polyps, asthma, and chronic sinusitis are aspirin-sensitive with no prior exposure to COX1 inhibitors.

ALOX15 (also known as 15-LO and 15-LOX) is a non-heme iron-containing dioxygenase that catalyzes the stereo-specific peroxidation of free and esterified polyunsaturated fatty acids generating a spectrum of bioactive lipid mediators. Additionally, ALOX15 converts arachidonic acid into 12-hydroperoxyeicosatetraenoic acid/12-HPETE and 15-hydroperoxyeicosatetraenoic acid/15-HPETE. ALOX15 also converts linoleic acid to 13-hydroperoxyoctadecadienoic acid. ALOX15 may also act on (12S)-hydroperoxyeicosatetraenoic acid/(12S)-HPETE to produce hepoxilin A3.

### SUMMARY

The present disclosure provides methods of treating a patient having a respiratory disorder, the method comprising administering an ALOX15 inhibitor to the patient. In some embodiments, the patient has a nasal polyp, allergic rhinitis, asthma, and/or AERD.

The present disclosure also provides methods of treating a patient with a therapeutic agent that treats or inhibits a respiratory disorder, wherein the patient is suffering from a respiratory disorder, the method comprising the steps of: determining whether the patient has an ALOX15 predicted loss-of-function variant nucleic acid molecule encoding a human ALOX15 polypeptide by: obtaining or having obtained a biological sample from the patient; and performing or having performed a genotyping assay on the biological sample to determine if the patient has a genotype comprising the ALOX15 predicted loss-of-function variant nucleic acid molecule; and when the patient is ALOX15 reference, then administering or continuing to administer to the patient the therapeutic agent that treats or inhibits a respiratory disorder in a standard dosage amount, and administering to the patient an ALOX15 inhibitor; and when the patient is heterozygous for an ALOX15 predicted loss-of-function variant, then administering or continuing to administer to the patient the therapeutic agent that treats or inhibits a respiratory disorder in an amount that is the same as or lower than a standard dosage amount, and administering to the patient an ALOX15 inhibitor; wherein the presence of a genotype having the ALOX15 predicted loss-of-function variant nucleic acid molecule encoding the human ALOX15 polypeptide indicates the patient has a reduced risk of developing a respiratory disorder.

The present disclosure also provides methods of identifying a human subject having an increased risk for developing a respiratory disorder, wherein the method comprises: determining or having determined the presence or absence of an ALOX15 predicted loss-of-function variant nucleic acid molecule encoding a human ALOX15 polypeptide in a biological sample obtained from the subject; wherein: when the human subject is ALOX15 reference, then the human subject has an increased risk for developing a respiratory disorder; and when the human subject is heterozygous for an ALOX15 predicted loss-of-function variant or homozygous for an ALOX15 predicted loss-of-function variant, then the human subject has a decreased risk for developing a respiratory disorder.

The present disclosure also provides methods of detecting an ALOX15 variant nucleic acid molecule in a human subject comprising assaying a sample obtained from the human subject to determine whether a nucleic acid molecule in the sample is: a genomic nucleic acid molecule comprising a nucleotide sequence comprising a thymine at a position corresponding to position 9,917 according to SEQ ID NO:2, or the complement thereof; an mRNA molecule comprising a nucleotide sequence comprising a uracil at a position corresponding to position 1,693 according to SEQ ID NO:4, or the complement thereof; or a cDNA molecule comprising a nucleotide sequence comprising a thymine at a position corresponding to position 1,693 according to SEQ ID NO:6, or the complement thereof.

The present disclosure also provides methods of detecting the presence of a human ALOX15 Thr560Met variant polypeptide, comprising performing an assay on a sample obtained from a human subject to determine whether an ALOX15 protein in the sample comprises a methionine at a position corresponding to position 560 according to SEQ ID NO:8.

The present disclosure also provides therapeutic agents that treat or inhibit a respiratory disorder for use in the treatment of a respiratory disorder in a human subject having: a genomic nucleic acid molecule having a nucleotide sequence encoding a human ALOX15 polypeptide, wherein the nucleotide sequence comprises a thymine at a position corresponding to position 9,917 according to SEQ ID NO:2, or the complement thereof; an mRNA molecule having a nucleotide sequence encoding a human ALOX15 polypeptide, wherein the nucleotide sequence comprises a uracil at a position corresponding to position 1,693 according to SEQ ID NO:4, or the complement thereof; or a cDNA molecule having a nucleotide sequence encoding a human ALOX15 polypeptide, wherein the nucleotide sequence comprises a thymine at a position corresponding to position 1,693 according to SEQ ID NO: 6, or the complement thereof.

The present disclosure also provides ALOX15 inhibitors for use in the treatment of a respiratory disorder in a human subject having: a genomic nucleic acid molecule having a nucleotide sequence encoding a human ALOX15 polypeptide, wherein the nucleotide sequence comprises a thymine at a position corresponding to position 9,917 according to SEQ ID NO:2, or the complement thereof; an mRNA molecule having a nucleotide sequence encoding a human ALOX15 polypeptide, wherein the nucleotide sequence comprises a uracil at a position corresponding to position 1,693 according to SEQ ID NO:4, or the complement thereof; or a cDNA molecule having a nucleotide sequence encoding a human ALOX15 polypeptide, wherein the nucleotide sequence comprises a thymine at a position corresponding to position 1,693 according to SEQ ID NO:6, or the complement thereof.

### BRIEF DESCRIPTION OF THE DRAWINGS

The accompanying figures, which are incorporated in and constitute a part of this specification, illustrate several features of the present disclosure.

FIG. 1 shows a table that indicates that rs34210653 significantly associates with reduced eosinophils in UK Biobank 50K exomes.

FIG. 2 shows a table that indicates that rs34210653 significantly associates with reduced eosinophils in UK Biobank 500K genotyping.

FIG. 3 shows a table that indicates that rs34210653 significantly associates with decreased odds of nasal polyp and allergic rhinitis and associates with doctor diagnosed asthma in UK Biobank 500K genotyping.

FIG. 4 shows a table that indicates that rs34210653 significantly associates with decreased eosinophils in GHS 90K exomes.

FIG. 5 shows a table that indicates that ALOX15 associates with decreased odds of nasal polyp in GHS 90K exomes.

FIG. 6 shows a locus zoom plot for the significant association between rs34210653 and eosinophils in UKB 500K genotyped data.

FIG. 7 shows quantitative trait distributions for eosinophil counts in UKB 500K genotyping data showing decreased eosinophils among heterozygous and homozygous rs34210653 variant carriers (dotted line indicates mean eosinophil numbers for ALOX15 reference allele carriers).

FIG. 8 shows a locus zoom plot for the significant association between rs34210653 and nasal polyps in UKB 500K genotyped data.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

A rare variant in the ALOX15 gene associated with a decreased risk of developing a respiratory disorder, such as nasal polyps, allergic rhinitis, asthma, and AERD, in human subjects has been identified in accordance with the present disclosure. For example, a genetic alteration (rs34210653) that changes the cytosine nucleotide at position 9,917 in the human ALOX15 reference (see, SEQ ID NO:1) to thymine has been observed to indicate that the human having such an alteration may have a decreased risk of developing a respiratory disorder, such as nasal polyps, allergic rhinitis, asthma, and AERD. It is believed that no variants of the ALOX15 gene or protein have any known association with a respiratory disorder, such as nasal polyps, allergic rhinitis, asthma, and AERD. Altogether, the genetic analyses described herein surprisingly indicate that the ALOX15 gene and, in particular, a variant in the ALOX15 gene, associates with a decreased risk of developing a respiratory disorder, such as nasal polyps, allergic rhinitis, asthma, and AERD. Therefore, human subjects that are ALOX15 reference that have an increased risk of developing a respiratory disorder, such as nasal polyps, allergic rhinitis, asthma, and/or AERD, may be treated such that a respiratory disorder is prevented, the symptoms thereof are reduced, and/or development of symptoms is repressed. Accordingly, the present disclosure provides methods of leveraging the identification of such variants in subjects to identify or stratify risk in such subjects of developing a respiratory disorder, such as nasal polyps, allergic rhinitis, asthma, and/or AERD, or to diagnose subjects as having an increased risk of developing a respiratory disorder, such as nasal polyps, allergic rhinitis, asthma, and/or AERD, such that subjects at risk or subjects with active disease may be treated accordingly.

For purposes of the present disclosure, any particular human can be categorized as having one of three ALOX15 genotypes: i) ALOX15 reference; ii) heterozygous for an ALOX15 predicted loss-of-function variant; or iii) homozygous for an ALOX15 predicted loss-of-function variant. A human is ALOX15 reference when the human does not have a copy of an ALOX15 predicted loss-of-function variant nucleic acid molecule. A human is heterozygous for an ALOX15 predicted loss-of-function variant when the human has a single copy of an ALOX15 predicted loss-of-function variant nucleic acid molecule. An ALOX15 predicted loss-of-function variant nucleic acid molecule is any ALOX15 nucleic acid molecule (such as, a genomic nucleic acid molecule, an mRNA molecule, or a cDNA molecule) encoding an ALOX15 polypeptide having a partial loss-of-function, a complete loss-of-function, a predicted partial loss-of-function, or a predicted complete loss-of-function. A human who has an ALOX15 polypeptide having a partial loss-of-function (or predicted partial loss-of-function) is hypomorphic for ALOX15. The ALOX15 predicted loss-of-function variant nucleic acid molecule can be any nucleic acid molecule encoding ALOX15 Thr560Met, Tyr139Cys, Leu651fs, Pro565Leu, Asn658Lys, Gly283Arg, Val474Ala, Gly422Arg, or Leu106fs. In some embodiments, the ALOX15 predicted loss-of-function variant nucleic acid molecule encodes ALOX15 Thr560Met. A human is homozygous for an ALOX15 predicted loss-of-function variant when the human has two copies of an ALOX15 predicted loss-of-function variant nucleic acid molecule.

For human subjects or patients that are genotyped or determined to be ALOX15 reference, such human subjects or patients have an increased risk of developing a respiratory disorder, such as nasal polyps, allergic rhinitis, asthma, and/or AERD. For human subjects or patients that are genotyped or determined to be either ALOX15 reference or heterozygous for an ALOX15 predicted loss-of-function variant, such human subjects or patients can be treated with an ALOX15 inhibitor.

In any of the embodiments described herein, the ALOX15 predicted loss-of-function variant nucleic acid molecule can be any ALOX15 nucleic acid molecule (such as, for example, genomic nucleic acid molecule, mRNA molecule, or cDNA molecule) encoding an ALOX15 polypeptide having a partial loss-of-function, a complete loss-of-function, a predicted partial loss-of-function, or a predicted complete loss-of-function. For example, the ALOX15 predicted loss-of-function variant nucleic acid molecule can be any nucleic acid molecule encoding ALOX15 Thr560Met, Tyr139Cys, Leu651fs, Pro565Leu, Asn658Lys, Gly283Arg, Val474Ala, Gly422Arg, or Leu106fs. In some embodiments, the ALOX15 predicted loss-of-function variant nucleic acid molecule encodes ALOX15 Thr560Met.

In any of the embodiments described herein, the ALOX15 predicted loss-of-function polypeptide can be any ALOX15 polypeptide having a partial loss-of-function, a complete loss-of-function, a predicted partial loss-of-function, or a predicted complete loss-of-function. In any of the embodiments described herein, the ALOX15 predicted loss-of-function polypeptide can be any of the ALOX15 polypeptides described herein including, for example, ALOX15 Thr560Met, Tyr139Cys, Leu651fs, Pro565Leu, Asn658Lys, Gly283Arg, Val474Ala, Gly422Arg, or Leu106fs. In some embodiments, the ALOX15 predicted loss-of-function polypeptide is ALOX15 Thr560Met.

In any of the embodiments described herein, the respiratory disorder is a nasal polyp, allergic rhinitis, asthma, and/or AERD. In any of the embodiments described herein, the respiratory disorder is a nasal polyp. In any of the embodiments described herein, the respiratory disorder is allergic rhinitis. In any of the embodiments described herein, the respiratory disorder is asthma. In any of the embodiments described herein, the respiratory disorder is AERD.

The present disclosure provides methods of treating a patient having a respiratory disorder, the methods comprising administering an ALOX15 inhibitor to the patient.

The present disclosure also provides methods of treating a patient having a nasal polyp, the methods comprising administering an ALOX15 inhibitor to the patient.

The present disclosure also provides methods of treating a patient having allergic rhinitis, the methods comprising administering an ALOX15 inhibitor to the patient. In some embodiments, the allergic rhinitis is high-eosinophil allergic rhinitis.

The present disclosure also provides methods of treating a patient having asthma, the methods comprising administering an ALOX15 inhibitor to the patient. In some embodiments, the asthma is allergic asthma, moderate-to-severe asthma, oral corticosteroid dependent asthma, eosinophilic asthma, or high-eosinophil eosinophilic asthma. In some embodiments, the asthma is allergic asthma. In some embodiments, the asthma is moderate-to-severe asthma. In some embodiments, the asthma is oral corticosteroid dependent asthma. In some embodiments, the asthma is eosinophilic asthma. In some embodiments, the asthma is high-eosinophil eosinophilic asthma. In some embodiments, the asthma is eosinophilic asthma-Chronic Obstructive Pulmonary Disease overlap syndrome (ACOS). In some embodiments, the ACOS is high-eosinophil eosinophilic ACOS.

The present disclosure also provides methods of treating a patient having AERD, the methods comprising administering an ALOX15 inhibitor to the patient.

The present disclosure also provides methods of treating a patient having atopy, the methods comprising administering an ALOX15 inhibitor to the patient. In some embodiments, the atopy is allergic rhinitis, asthma, and/or atopic dermatitis.

In some embodiments, the respiratory disorder is chronic obstructive pulmonary disease (COPD), chronic bronchitis, emphysema, allergic pneumonia, and/or an allergic airway disease.

In some embodiments, the ALOX15 inhibitor comprises an antisense molecule. Examples of antisense molecules include, but are not limited to, antisense nucleic acid molecules, small interfering RNAs (siRNAs), and short hairpin RNAs (shRNAs). Such antisense molecules can be designed to target any region of an ALOX15 mRNA. In some embodiments, the antisense RNA, siRNA, or shRNA hybridizes to a sequence within an ALOX15 genomic nucleic acid molecule or mRNA molecule and decreases expression of the ALOX15 polypeptide in a cell in the subject. In some embodiments, the ALOX15 inhibitor comprises an antisense RNA that hybridizes to an ALOX15 genomic nucleic acid molecule or mRNA molecule and decreases expression of the ALOX15 polypeptide in a cell in the subject. In some embodiments, the ALOX15 inhibitor comprises an siRNA that hybridizes to an ALOX15 genomic nucleic acid molecule or mRNA molecule and decreases expression of the ALOX15 polypeptide in a cell in the subject. In some embodiments, the ALOX15 inhibitor comprises an shRNA that hybridizes to an ALOX15 genomic nucleic acid molecule or mRNA molecule and decreases expression of the ALOX15 polypeptide in a cell in the subject. In some embodiments, the shRNA comprises: a) CCGGGAAACTGGAAGGACGGGTTAACTCGAGTTAACC CGTCCTTCCAGTTTCTTTTTTG (SEQ ID NO:9); b) CCGGGCTATCAAAGACTCTCTAAATCTCGAGAT TTAGAGAGTCTTTGATAGCTTTTTG (SEQ ID NO:10); c) CCGGTGGGAAATCATCTATCGGTATCTCG AGATACCGATAGATGATITCCCATTTTTG (SEQ ID NO:11); d) CCGGCCTGGAAGGAAGATGCCTTA TCTCGAGATAAGGCATCTTCCTTCCAGGTTTTTG (SEQ ID NO:12); e) CCGGCCAGTTTCTTAATGGC GCCAACTCGAGTTGGCGCCATTAAGAAACTGGTTTTTG (SEQ ID NO:13); f) CCGGGCCGTCGATAC ATCCTATCTTCTCGAGAAGATAGGATGTATCGACGGCTTTTTG (SEQ ID NO:14); g) CCGGTAGATG ACTTCAACCGGATTTCTCGAGAAATCCGGTTGAAGTCATCTATTTTTTG (SEQ ID NO:15); or h) CCG GTGGTACTCTTGGGTGCCTAATCTCGAGATTAGGCACCCAAGAGTACCATTTTTTG (SEQ ID NO:16).

In some embodiments, the ALOX15 inhibitor comprises a nuclease agent that induces one or more nicks or double-strand breaks at a recognition sequence(s) or a DNA-binding protein that binds to a recognition sequence within an ALOX15 genomic nucleic acid molecule. The recognition sequence can be located within a coding region of the ALOX15 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ALOX15 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an ALOX15 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of ALOX15 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an ALOX15 genomic nucleic acid molecule or it can be a nickase that creates a single-strand break in an ALOX15 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of ALOX15 genomic nucleic acid molecules can be generated by contacting a cell with a Cas protein and one or more gRNAs that hybridize to one or more gRNA recognition sequences within a target genomic locus in the ALOX15 genomic nucleic acid molecule. For example, a gRNA recognition sequence can be located within a region of SEQ ID NO:1. The gRNA recognition sequence can also include or be proximate to a position corresponding to position 9,917 according to SEQ ID NO:1. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 9,917 according to SEQ ID NO:1. The gRNA recognition sequence can include or be proximate to the start codon of an ALOX15 genomic nucleic acid molecule or the stop codon of an ALOX15 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an ALOX15 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where Nis any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an ALOX15 genomic nucleic acid molecule. An exemplary gRNA is a gRNA effective to direct a Cas enzyme to bind to or cleave an ALOX15 genomic nucleic acid molecule, wherein the gRNA comprises a DNA-targeting segment that hybridizes to a gRNA recognition sequence within the ALOX15 genomic nucleic acid molecule that includes or is proximate to a position corresponding to position 9,917. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of a position corresponding to position 9,917 according to SEQ ID NO:1. Other exemplary gRNAs comprise a DNA-targeting segment that hybridizes to a gRNA recognition sequence present within an ALOX15 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human ALOX15 reference gene are set forth in Table 1 as SEQ ID NOS: 19-43.

TABLE 1
Guide RNA Recognition Sequences Near ALOX15
Variation(s)

| Strand | Guide RNA Recognition Sequence | SEQ ID NO: |
|---|---|---|
| − | TACCAAGCACGCGAGCCCCGTGG | 19 |
| + | GACCACGGGGCTCGCGTGCTTGG | 20 |
| + | ACGGGGCTCGCGTGCTTGGTAGG | 21 |
| − | GCATCCTTGGTGGTTGGCGGGGG | 22 |
| − | CGGGGGCAGCCGCATCGTGCAGG | 23 |
| + | TTAGCTGGACTGGTACTCTTGGG | 24 |
| − | GGGGGCAGCCGCATCGTGCAGGG | 25 |
| + | CTTAGCTGGACTGGTACTCTTGG | 26 |
| − | TGCATCCTTGGTGGTTGGCGGGG | 27 |
| − | CAAGAGTACCAGTCCAGCTAAGG | 28 |
| + | GATGTCCATCACTTGGCAGCTGG | 29 |
| − | GTACCAGTCCAGCTAAGGAAGGG | 30 |
| − | TTGCATCCTTGGTGGTTGGCGGG | 31 |
| + | CTGTGTGCAGGTGGACCACGGGG | 32 |
| + | TCTGTGTGCAGGTGGACCACGGG | 33 |
| + | TGCTTGGTAGGCACTGACTCTGG | 34 |
| − | AGTACCAGTCCAGCTAAGGAAGG | 35 |
| + | CTGCCCTTCCTTAGCTGGACTGG | 36 |
| + | GACACTGCCCAACTTCCACCAGG | 37 |
| − | AGAGAAGCCTGGTGGAAGTTGGG | 38 |
| + | GATCCCTGCCCTTCCTTAGCTGG | 39 |
| + | GTCTGTGTGCAGGTGGACCACGG | 40 |
| − | CAGTCCAGCTAAGGAAGGGCAGG | 41 |
| − | GCTGCCAAGTGATGGACATCTGG | 42 |
| − | TCTGCCCAGCTGCCAAGTGATGG | 43 |

The Cas protein and the gRNA form a complex, and the Cas protein cleaves the target ALOX15 genomic nucleic acid molecule. The Cas protein can cleave the nucleic acid molecule at a site within or outside of the nucleic acid sequence present in the target ALOX15 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the ALOX15 genomic nucleic acid molecule to which a DNA-targeting segment of a gRNA will bind.

Such methods can result, for example, in an ALOX15 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ALOX15 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the ALOX15 inhibitor comprises a small molecule. In some embodiments, the ALOX15 inhibitor is 6,11-dihydro[1]benzothiopyrano[4,3-b]indole (PD146176), 2-bromophenol, 2,4-dibromophenol, 2-(1-thienyl)ethyl-3,4-dihydroxybenzylidenecyanoacetate (TEDC), 4,4′-(2,3-dimethyl-1,4-butanediyl)bis-1,2-benzenediol (nordihydroguaiaretic acid), or cinnamyl-3,4-dihydroxy-a-cyanocinnamate (CDC). In some embodiments, the ALOX15 inhibitor is 2-bromophenol, 2,4-dibromophenol, 2-(1-thienyl)ethyl-3,4-dihydroxybenzylidenecyanoacetate (TEDC), or 4,4′-(2,3-dimethyl-1,4-butanediyl)bis-1,2-benzenediol (nordihydroguaiaretic acid).

In some embodiments, the methods of treatment further comprise detecting the presence or absence of an ALOX15 predicted loss-of-function variant nucleic acid molecule encoding a human ALOX15 polypeptide in a biological sample from the patient. As used throughout the present disclosure, a “ALOX15 predicted loss-of-function variant nucleic acid molecule” is any ALOX15 nucleic acid molecule (such as, for example, genomic nucleic acid molecule, mRNA molecule, or cDNA molecule) encoding an ALOX15 polypeptide having a partial loss-of-function, a complete loss-of-function, a predicted partial loss-of-function, or a predicted complete loss-of-function.

The present disclosure also provides methods of treating a patient with a therapeutic agent that treats or inhibits a respiratory disorder, wherein the patient is suffering from a respiratory disorder. In some embodiments, the methods comprise determining whether the patient has an ALOX15 predicted loss-of-function variant nucleic acid molecule encoding a human ALOX15 polypeptide by obtaining or having obtained a biological sample from the patient, and performing or having performed a genotyping assay on the biological sample to determine if the patient has a genotype comprising the ALOX15 predicted loss-of-function variant nucleic acid molecule. When the patient is ALOX15 reference, the therapeutic agent that treats or inhibits a respiratory disorder is administered or continued to be administered to the patient in a standard dosage amount, and an ALOX15 inhibitor is administered to the patient. When the patient is heterozygous for an ALOX15 predicted loss-of-function variant, the therapeutic agent that treats or inhibits a respiratory disorder is administered or continued to be administered to the patient in an amount that is the same as or lower than a standard dosage amount, and an ALOX15 inhibitor is administered to the patient. The presence of a genotype having the ALOX15 predicted loss-of-function variant nucleic acid molecule encoding the human ALOX15 polypeptide indicates the patient has a reduced risk of developing a respiratory disorder. In some embodiments, the patient is ALOX15 reference. In some embodiments, the patient is heterozygous for an ALOX15 predicted loss-of-function variant.

For human subjects or patients that are genotyped or determined to be either ALOX15 reference or heterozygous for an ALOX15 predicted loss-of-function variant, such human subjects or patients can be treated with an ALOX15 inhibitor, as described herein.

Detecting the presence or absence of an ALOX15 predicted loss-of-function variant nucleic acid molecule in a biological sample from a patient and/or determining whether a patient has an ALOX15 predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the human subject.

In some embodiments, when the patient is ALOX15 reference, the patient is also administered a therapeutic agent that treats or inhibits a respiratory disorder in a standard dosage amount. In some embodiments, when the patient is heterozygous for an ALOX15 predicted loss-of-function variant, the patient is also administered a therapeutic agent that treats or inhibits a respiratory disorder in a dosage amount that is the same as or lower than a standard dosage amount.

In some embodiments, the treatment methods further comprise detecting the presence or absence of an ALOX15 predicted loss-of-function polypeptide in a biological sample from the patient. In some embodiments, when the patient does not have an ALOX15 predicted loss-of-function polypeptide, the patient is also administered a therapeutic agent that treats or inhibits a respiratory disorder in a standard dosage amount. In some embodiments, when the patient has an ALOX15 predicted loss-of-function polypeptide, the patient is also administered a therapeutic agent that treats or inhibits a respiratory disorder in a dosage amount that is the same as or lower than a standard dosage amount.

The present disclosure also provides methods of treating a patient with a therapeutic agent that treats or inhibits a respiratory disorder, wherein the patient is suffering from a respiratory disorder. In some embodiments, the method comprises determining whether the patient has an ALOX15 predicted loss-of-function polypeptide by obtaining or having obtained a biological sample from the patient, and performing or having performed an assay on the biological sample to determine if the patient has an ALOX15 predicted loss-of-function polypeptide. When the patient does not have an ALOX15 predicted loss-of-function polypeptide, the therapeutic agent that treats or inhibits a respiratory disorder is administered or continued to be administered to the patient in a standard dosage amount, and an ALOX15 inhibitor is administered to the patient. When the patient has an ALOX15 predicted loss-of-function polypeptide, the therapeutic agent that treats or inhibits a respiratory disorder is administered or continued to be administered to the patient in an amount that is the same as or lower than a standard dosage amount, and an ALOX15 inhibitor is administered to the patient. The presence of an ALOX15 predicted loss-of-function polypeptide indicates the patient has a reduced risk of developing a respiratory disorder. In some embodiments, the patient has an ALOX15 predicted loss-of-function polypeptide. In some embodiments, the patient does not have an ALOX15 predicted loss-of-function polypeptide.

Detecting the presence or absence of an ALOX15 predicted loss-of-function polypeptide in a biological sample from a patient and/or determining whether a patient has an ALOX15 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the human subject.

Examples of therapeutic agents that treat or inhibit a respiratory disorder include, but are not limited to: steroidal anti-inflammatory agents, non-steroidal anti-inflammatory agents, anti-proliferative agents, anti-macrophage agents, muscle relaxants, narcotic analgesics, non-narcotic analgesics, local anesthetic agents, and miscellaneous agents. An additional example of a therapeutic agent that treats or inhibits the respiratory disorder is an interleukin-4 receptor (IL-4Ra) antagonist such as, for example, DUPIXENT® (dupilumab).

Examples of steroidal anti-inflammatory agents include, but are not limited to, glucocorticoids and corticosteroids such as, for example, 21-acetoxypregnenolone, alclometasone, algestone, amicinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumehtasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone, formocortal, halcinonide, halobetasol priopionate, halometasone, halopredone acetate, hydrocortamate, hydrocortisone, loteprednol etabonate, mazipredone, medrysone, meprednisone, methyolprednisolone, mometasone furoate, paramethasone, prednicarbate, prednisolone, prednisolone 25-diethylaminoacetate, prednisone sodium phosphate, prednisone, prednival, prednylidene, rimexolone, tixocortal, triamcinolone, triamcinolone acetonide, triamcinolone benetonide, and triamcinolone hexacetonide.

Examples of non-steroidal anti-inflammatory agents include, but are not limited to, aminoarylcarboxylic acid derivatives such as, for example, enfenamic acid, etofenamate, flufenamic acid, isonixin, meclofenamic acid, mefanamic acid, niflumic acid, talniflumate, terofenamate, and tolfenamic acid; arylacetic acid derivatives such as, for example, acemetacin, alclofenac, amfenac, bufexamac, cinmetacin, clopirac, diclofenac, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin, and zomepirac; arylbutyric acid derivatives such as, for example, bumadizon, butibufen, fenbufen, and xenbucin; arylcarboxylic acids such as, for example, clidanac, ketorolac, and tinoridine; arylpropionic acid derivatives such as, for example, alminoprofen, benoxaprofen, bucloxic acid, carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen, and tiaprofenic acid; pyrazoles such as, for example, difenamizole and epirizole; pyrazolones such as, for example, apazone, benzpiperylon, feprazone, mofebutazone, morazone, oxyphenbutazone, phenybutazone, pipebuzone, propyphenazone, ramifenazone, suxibuzone, and thiazolinobutazone; salicylic acid and its derivatives such as, for example, acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate, and sulfasalazine; thiazinecarboxamides such as, for example, droxicam, isoxicam, piroxicam, and tenoxicam; and other agents such as, for example, ¿-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

Examples of anti-proliferative agents include, but are not limited to, anthracyclines, alkyl sulfonates, agents affecting microtubule dynamics, agents affecting various growth factors including IGF pathway agents such as somatostatin analogs, angiotensin converting enzyme inhibitors, antimetabolites (e.g., purine analogs), cytotoxic agents, cytostatic agents, cell proliferation affectors, caspase activators, proteasome inhibitors, angiogenesis inhibitors, ethylenimines, intercalating agents, metal coordination complexes, nitrogen mustards, nitrosoureas, nucleic acid damaging agents such as alkylating agents, purine analogs, pyrimidine analogs, inhibitors of pyrimidine biosynthesis, vinca alkaloids. Additional examples include, but are not limited to, adriamycin, alitretinoin (9-cis-retinoic acid), amifostine, angiopeptin, angiostatin, arabinosyl 5-azacytosine, arabinosyl cytosine, 5-aza-2′-deoxycytidine, 6-azacytidine, 6-azauridine, azaribine, bexarotene (4-[1-(5,6,7,8-tetrahydro-3,5,5,8,8-pentamethyl-2-naphthalenyl)ethenyl]benzoic acid), bleomycin, BCNU, CCNU, captopril, capecitabine (5′-deoxy-5-fluoro-cytidine), chlorambucil, coichicine, cilazapril, cisplatin, cladribine (a chlorinated purine nucleoside analog), cytarabine, cyclocytidine, cyclophosphamide, daunorubicin, 3-deazauridine, 2′-deoxy-5-fluorouridine, 5′-deoxy-5-fluorouridine, docetaxel, doxorubicin, endostatin, epirubicin, epothilone, estramustine, etoposide, exemestane, flutamide, fludarabine, fludarabin phosphate, fluorocytosine, 5-fluorouracil, 5-fluorouridine, 5-fluoro-2′-deoxyuridine, gemcitabine, hydroxyurea, idarubicin, irinotecan, LHRH analogs, lisinopril, melphalan, methotrexate, 6-mercaptopurine, mitoxantrone, ocreotide, paclitaxel, pentostatin, N-phosphonoacetyl-L-aspartic acid, prednimustine, pyrazofurin, squalamine, streptozocin, tamoxifen, temozolomide, teniposide, 6-thioguanine, tomudex, thiotepa, topotecan, 5-trifluoromethyl-2′-deoxyuridine, valrubicin, vincristine, vinblastine, and vinarelbine.

Examples of anti-macrophage agents include, but are not limited to, bisphosphonates and chlodronate compounds such as, for example, dichloromethylene diphosphonate (CL2MDP).

Examples of muscle relaxants include, but are not limited to, anticholinergics (for example, adiphenine, alverine, ambutonomium, aminopentamide, amixetrine, amprotropine phosphate, anisotropine methylbromide, apoatropine, atropine, atropine n-oxide, benactyzine, benapryzine, benzetimide, benzilonium, benztropine mesylate, bevonium methyl sulfate, biperiden, butropium, n-butylscopolammonium bromide, buzepide, camylofine, caramiphen, chlorbenzoxamine, chlorphenoxamine, cimetropium, clidinium, cyclodrine, cyclonium, cycrimine, deptropine, dexetimide, dibutoline sulfate, dicyclomine, diethazine, difemerine, dihexyverine, diphemanil methylsulfate, n-(1,2-diphenylethyl) nicotinamide, dipiproverine, diponium, emepronium, endobenzyline, ethopropazine, ethybenztropine, ethylbenzhydramine, etomidoline, eucatropine, fenpiverinium, fentonium, flutropium, glycopyrrolate, heteronium, hexocyclium methyl sulfate, homatropine, hyoscyamine, ipratropium, isopropamide, levomepate, mecloxamine, mepenzolate, metcaraphen, methantheline, methixene, methscopolamine, octamylamine, oxybutynin, oxyphencyclimine, oxyphenonium, pentapiperide, penthienate, phencarbamide, phenglutarimide, pipenzolate, piperidolate, piperilate, poldine methysulfate, pridinol, prifinium, procyclidine, propantheline, propenzolate, propiverine, propyromazine, scopolamine, scopolamine n-oxide, stramonium, sultroponium, thiphenamil, tiemonium, timepidium, tiquizium, tridihexethyl iodide, trihexyphenidyl hydrochloride, trimebutine, tropacine, tropenzile, tropicamide, trospium, valethamate, vamicamide, and xenytropium); alcuronium, atracurium, baclofen, benzodiazepines (for example, clozapine or diazepam), botulinum toxin (BOTOX), 4-amino-3-(4-chloropheyl)-butanoic acid, carbolonium, carisoprodol, chlorphenesin, chlorzoxazone, cyclobenzaprine, cyclandelate, dantrolene, decamethonium bromide, diazepam hydralazine, fazadinium, gallamine, guaifenesin, hexafluorenium, isoxsuprine, meladrazine, mephensin, metaxalone, methocarbamol nylidrin, metocurine iodide, orphenadrine, pancuronium, papaverine, pridinol, styramate, suxamethonium, suxethonium, thiocolchicoside, tizanidine, suxamethonium, tolperisone, and tubocurarine; brochodilators (ephedrine derivatives such as, for example, albuterol, bambuterol, bitolterol, carbuterol, clenbuterol, clorprenaline, dioxethedrine, ephedrine, epiniphrine, eprozinol, etafedrine, ethylnorepinephrine, fenoterol, hexoprenaline, isoetharine, isoproterenol, mabuterol, metaproterenol, n-methylephedrine, pirbuterol, procaterol, protokylol, reproterol, rimiterol, salmeterol, soterenol, terbutaline, and tulobuterol; quaternary ammonium compounds such as, for example, bevonium methyl sulfate, clutropium bromide, ipratropium bromide; and oxitropium bromide; xanthine derivatives such as, for example, acefylline, acefylline piperazine, ambuphylline, aminophylline, bamifylline, choline theophyllinate, doxofylline, dyphylline, enprofylline, etamiphyllin, etofylline, guaithylline, proxyphylline, theobromine, 1-theobromineacetic acid, and theophylline; and other bronchodilators such as, for example, fenspiride, medibazine, montekulast, methoxyphenanime, tretoquinol, zafirkulast, and cathcholamine analogs such as formoterol); antispasmodics (for example, alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, coniine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, n,n-1trimethyl-3,3-diphenyl-propylamine, tropenzile, trospium chloride, and xenytropium bromide); and anticholinergics (for example, adiphenine, alverine, ambutonomium, aminopentamide, amixetrine, amprotropine phosphate, anisotropine methylbromide, apoatropine, atropine, atropine n-oxide, benactyzine, benapryzine, benzetimide, benzilonium, benztropine mesylate, bevonium methyl sulfate, biperiden, butropium, n-butylscopolammonium bromide, buzepide, camylofine, caramiphen, chlorbenzoxamine, chlorphenoxamine, cimetropium, clidinium, cyclodrine, cyclonium, cycrimine, deptropine, dexetimide, dibutoline sulfate, dicyclomine, diethazine, difemerine, dihexyverine, diphemanil methylsulfate, n-(1,2-diphenylethyl) nicotinamide, dipiproverine, diponium, emepronium, endobenzyline, ethopropazine, ethybenztropine, ethylbenzhydramine, etomidoline, eucatropine, fenpiverinium, fentonium, flutropium, glycopyrrolate, heteronium, hexocyclium methyl sulfate, homatropine, hyoscyamine, ipratropium, isopropamide, levomepate, mecloxamine, mepenzolate, metcaraphen, methantheline, methixene, methscopolamine, octamylamine, oxybutynin, oxyphencyclimine, oxyphenonium, pentapiperide, penthienate, phencarbamide, phenglutarimide, pipenzolate, piperidolate, piperilate, poldine methysulfate, pridinol, prifinium, procyclidine, propantheline, propenzolate, propiverine, propyromazine, scopolamine, scopolamine n-oxide, stramonium, sultroponium, thiphenamil, tiemonium, timepidium, tiquizium, tridihexethyl iodide, trihexyphenidyl hydrochloride, trimebutine, tropacine, tropenzile, tropicamide, trospium, valethamate, vamicamide, and xenytropium).

Examples of narcotic analgesics include, but are not limited to, alfentanil, allylprodine, alphaprodine, anileridine, benzylmorphine, bezitramide, buprenorphine, butorphanol, clonitazene, codeine, codeine methyl bromide, codeine phosphate, codeine sulfate, desomorphine, dextromoramide, dezocine, diampromide, dihydrocodeine, dihydrocodeinone enol acetate, dihydromorphine, dimenoxadol, dimepheptanol, dimethylthiambutene, dioxaphetyl butyrate, dipipanone, eptazocine, ethoheptazine, ethylmethlythiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone hydrochloride, metopon, morphine, myrophine, nalbuphine, narceine, nicomorphine, norlevorphanol, normethadone, normorphine, norpipanone, opium, oxycodone, oxymorphone, papaveretum, pentazocine, phenadoxone, phenazocine, pheoperidine, piminodine, piritramide, proheptazine, promedol, properidine, propiram, propoxyphene, rumifentanil, sufentanil, and tilidine.

Examples of non-narcotic analgesics include, but are not limited to, aceclofenac, acetaminophen, acetaminosalol, acetanilide, acetylsalicylsalicylic acid, alclofenac, alminoprofen, aloxiprin, aluminum bis(acetylsalicylate), aminochlorthenoxazin, 2-amino-4-picoline, aminopropylon, aminopyrine, ammonium salicylate, amtolmetin guacil, antipyrine, antipyrine salicylate, antrafenine, apazone, aspirin, benorylate, benoxaprofen, benzpiperylon, benzydamine, bermoprofen, brofenac, p-bromoacetanilide, 5-bromosalicylic acid acetate, bucetin, bufexamac, bumadizon, butacetin, calcium acetylsalicylate, carbamazepine, carbiphene, carsalam, chloralantipyrine, chlorthenoxazin (e), choline salicylate, cinchophen, ciramadol, clometacin, cropropamide, crotethamide, dexoxadrol, difenamizole, diflunisal, dihydroxyaluminum acetylsalicylate, dipyrocetyl, dipyrone, emorfazone, enfenamic acid, epirizole, etersalate, ethenzamide, ethoxazene, etodolac, felbinac, fenoprofen, floctafenine, flufenamic acid, fluoresone, flupirtine, fluproquazone, flurbiprofen, fosfosal, gentisic acid, glafenine, ibufenac, imidazole salicylate, indomethacin, indoprofen, isofezolac, isoladol, isonixin, ketoprofen, ketorolac, p-lactophenetide, lefetamine, loxoprofen, lysine acetylsalicylate, magnesium acetylsalicylate, methotrimeprazine, metofoline, miroprofen, morazone, morpholine salicylate, naproxen, nefopam, nifenazone, 5′nitro-2′propoxyacetanilide, parsalmide, perisoxal, phenacetin, phenazopyridine hydrochloride, phenocoll, phenopyrazone, phenyl acetylsalicylate, phenyl salicylate, phenyramidol, pipebuzone, piperylone, prodilidine, propacetamol, propyphenazone, proxazole, quinine salicylate, ramifenazone, rimazolium metilsulfate, salacetamide, salicin, salicylamide, salicylamide o-acetic acid, salicylsulfuric acid, salsalte, salverine, simetride, sodium salicylate, sulfamipyrine, suprofen, talniflumate, tenoxicam, terofenamate, tetradrine, tinoridine, tolfenamic acid, tolpronine, tramadol, viminol, xenbucin, and zomepirac.

Examples of local anesthetic agents include, but are not limited to, amucaine, amolanone, amylocaine hydrochloride, benoxinate, benzocaine, betoxycaine, biphenamine, bupivacaine, butacaine, butaben, butanilicaine, butethamine, butoxycaine, carticaine, chloroprocaine hydrochloride, cocaethylene, cocaine, cyclomethycaine, dibucaine hydrochloride, dimethisoquin, dimethocaine, diperadon hydrochloride, dyclonine, ecgonidine, ecgonine, ethyl chloride, beta-eucaine, euprocin, fenalcomine, fomocaine, hexylcaine hydrochloride, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine hydrochloride, phenol, piperocaine, piridocaine, polidocanol, pramoxine, prilocaine, procaine, propanocaine, proparacaine, propipocaine, propoxycaine hydrochloride, pseudococaine, pyrrocaine, ropavacaine, salicyl alcohol, tetracaine hydrochloride, tolycaine, trimecaine, and zolamine.

Examples of miscellaneous agents include, but are not limited to, amlexanox, aminophylline, azelastine, beclometaason dipropionate, cromolyn, dexamethasone, ephedrine, fenoterol, flutropium, hydrocortisone, ibudilast, ipratropium, isoprenaline, ZAJITEN™ (ketotifen), leukotriene modifiers such as montelukast, zafirlukast and zileuton, ZESURAN™ (mequitazine), nedocromil, orciprenaline, oxitomide, oxitropium, pranlukast hydrate, prednisolone, procaterol, repirinast, salbutamol, seratrodast, sodium cromoglicate, suplatast tosylate, terbutaline, terfenadine, theophylline, tiaramide, tranilast, traxanox, trimetoquinol, and tubobuterol.

Examples of therapeutic agents that treats or inhibits the respiratory disorder that are suitable for use in treating nasal polyps include, but are not limited to: periostin antagonists, PP1 antagonists, MET antagonists, PIP agonists, and AZGP1 agonists. Examples of periostin antagonists include, but are not limited to, polyclonal antibodies against human periostin (BioVendor Laboratory Medicine, Inc, Modrice, Czech Republic, Cat. No.: RD-181045050) and anti-periostin monoclonal antibody (see, Tai et al., Carcinogenesis, 2005, 26, 908-15), and valsartan.

Examples of PP1 antagonists (such as, for example, PP1c antagonists, PPP1R9B antagonists, and PPP1R6 antagonists) include, but are not limited to, polyclonal antibodies against human neurabin 2 (also known as PPP1R9B) (Abcam, Inc, Cambridge, Mass., Cat. No.: AB18561), anti-PPP1R9B antibody (Novus Biological, Inc., Littleton, Co; Cat. No. H00084687-A01), inhibitory peptides specific for PP1 (such as, for example, a peptide having the amino acid sequence of MEPDNSPRKIQFTVPLLEPHLDPEAAEQIRRRRPTPATLVLTSDQSSPEI DEDRIPNSLLKSTLSMSPRQRKKMTRTTPTMKELQTMVEHHLGQQKQGEEPEGATESTGNQESCPPGI PDTGSASRPDTPGTAQKSAESNPKTQEQCGVEPRTEDSSAHMLPLDSQGASLV) (SEQ ID NO:17) and a peptide having the amino acid sequence of MAASTASHRPIKGILKNKTSSTSSRVASAEQPRGSV DEELSKKSQKWDEMNILATYHPADKDYGLMKIDEPSTPYHSMIGDDDDAYSDTETTEAMTPDTLAKKLA AAEGSEPKYRIREQESSGEEDSDLSPEEREKKRQFEMKRKLHYNEGLNIKLARQLISKDLHDDEEDEEM SETADGESMNTEESNQGSTPSDQRQNKSQSS (SEQ ID NO:18)); and dopamine- and cyclic AMP-regulated phosphoprotein (DARPP-32) (EMD Calbiochem, Inc., Gibbstown, N.J.; Cat. No. 251755); protein phosphatase inhibitors such as, for example, okadaic acid and Calyculin A.

Examples of MET antagonists include, but are not limited to, polyclonal antibodies against human MET (Santa Cruz Biotechnology, Inc, Santa Cruz, Calif., Cat. No.: SC-10) and anti-MET antibody (Sigma-Aldrich, St. Louis, Mo., Cat. No.: C7115); tyrosine kinase inhibitors such as, for example, (3Z)-N-(3-Chlorophenyl)-3-((3,5-dimethyl-4-((4-methylpiperazin-1-yl) carbonyl)-1H-pyrrol-2-yl)methylene)-N-methyl-2-oxo-2,3-dihydro-1H-indole-5-sulfonamide (SU11274) and 1,3-Dihydro-3-((3,5-dimethyl-1H-pyrrol-2-yl)methylene)-2H-indol-2-one (SU5416).

Examples of PIP agonists include, but are not limited to, PIP protein, recombinant PIP protein (Abnova, Taipei City, Taiwan, Cat. No.: H00005304-P01), and stimulators of PIP expression such as, for example, interleukin-4 and interleukin-13; and tamoxifen.

Examples of AZGP1 agonists include, but are not limited to, AZGP1 protein, recombinant AZGP1 protein (Abnova, Taipei City, Taiwan, Cat. No.: 1100000563-P01); rosiglitazone, dexamethasone, and (RR+SS)-(+)-4-(2-(2-(3-chlorophenyl)-2-hydroxyethyl)amino) propyl) phenoxyacetate (BRL 37344).

Examples of therapeutic agents that treats or inhibits the respiratory disorder that are suitable for use in treating allergic rhinitis include, but are not limited to: corticosteroids, H1 antagonist/antiallergic agents, histamine receptor antagonists, chemical mediator release inhibitors, thromboxane A2 receptor antagonists, thromboxane A2 synthesis inhibitors, leukotriene antagonists, Th2 cytokine inhibitors, TNF alpha antagonists, PDE4 inhibitors, JAK3 Inhibitors, and p38 Kinase Inhibitors.

Examples of corticosteroids include, but are not limited to, beclomethasone esters (KONAZE™, ARUDESHIN™, RHINOCORT™, SARUKOTO™ (17-propionate ester or the 17,21-dipropionate ester), Furunaze™ (fluticasone propionate), budesonide, flunisolide, mometasone esters (for example, furoate ester), triamcinolone acetonide, rofleponide, and ciclesonide.

Examples of H1 antagonist/antiallergic agents (antihistamines) include, but are not limited to, first generation H1 antihistamines such as, for example, piperoxam, ethylenediamine (for example, mepyramine (pyrilamine), antazoline), ethanolamines (for example, diphenhydramine, carbinoxamine, doxylamine, clemastine, and dimenhydrinate), alkylamines (for example, pheniramine, Kurorenamin (chlorpheniramine), dextrose cycloalkyl naphthoylmethyl Na Min, brompheniramine, and triprolidine), piperazines (for example, cyclizine, hydroxyzine, and meclizine), and tricyclics (for example, promethazine, alimemazine (trimeprazine), cyproheptadine, and azatadine); second-generation antihistamines such as, for example, systemic drugs (for example, acrivastine, astemizole, cetirizine, loratadine, mizolastine, and terfenadine), and local drugs (for example, azelastine, levocabastine, and olopatadine (Olopatidine); and third generation antihistamines such as, for example, (levocetirizine, desloratadine (desloratidin) and ALLEGRA® (fexofenadine).

Examples of histamine receptor antagonists include, but are not limited to, ZAJITEN™ (ketotifen), ZESURAN™ (mequitazine), ALLEGRA® (fexofenadine), EBASUTERU™ (ebastine), TALION™ (bepotastine), ALLELOCK™ (olopatadine), and CLARITIN® (loratadine).

Examples of chemical mediator release inhibitors include, but are not limited to, INTAL™ (cromoglycate) and RIZABEN™ (tranilast).

Examples of thromboxane A2 receptor antagonists include, but are not limited to, BRONICA™ (seratrodast) and BAINASU™ (ramatroban).

Examples of thromboxane A2 synthesis inhibitors include, but are not limited to, DOMENAN™ (ozagrel).

Examples of leukotriene antagonists include, but are not limited to, SINGULAIR® and KIPRES™ (montelukast) and ONON™ (pranlukast).

Examples of Th2 cytokine inhibitors include, but are not limited to, IPD™ (suplatast).

Examples of TNF alpha antagonists include, but are not limited to, ENBREL® (etanercept), REMICADE® (infliximab), and HUMIRA® (adalimumab).

Examples of PDE4 inhibitors include, but are not limited to, rolipram, piclamilast, CDP-840, ariflo, pentoxifylline, denbufylline, theophylline, substituted 8-arylquinoline phosphodiesterase-4 inhibitors (see, U.S. Pat. No. 6,740,666), alkyne-aryl phosphodiesterase-4 inhibitors (see, U.S. Pat. No. 6,743,802), 1-aryl-1,8-naphthyridin-4-one phosphodiesterase inhibitors (see, U.S. Pat. Nos. 6,677,351 and 6,541,480), hydroxyindoles (see, U.S. Pat. Nos. RE38,624, 6,613,794 and 6,602,890), phthalazine derivatives (see, U.S. Pat. No. 6,589,951), tricyclic phthalazine derivatives (see, U.S. Pat. No. 6,525,055), benzazine derivatives (see, U.S. Pat. No. 6,358,973), benzamides with tetrahydrofuranyloxy substituents (see, U.S. Pat. No. 6,303,789), diazepinoindolones (see, U.S. Pat. No. 6,239,130), 1-oxo-1-3-substituted phenyl-1,4-dihydro-1,8-naphthyridine-3-carboxamide phosphodiesterase-4 inhibitors (see, U.S. Patent App. Pub. No. 2006/0058316), N-substituted diarylamines (see, U.S. Patent App. Pub. No. 2005-0222207), allyne-aryl phosphodiesterase-4 inhibitors (see, U.S. Patent App. Pub. No. 2005-0070569), and naphthyridine derivatives (see, U.S. Patent App. Pub. No. 2004-0254212).

Examples of JAK3 Inhibitors include, but are not limited to, tacrolimus, CP-690550, WHI-P131, WHIP-97, WHIP-154, AG490, PS-608504, and PNU156804. Additional examples include, but are not limited to, 2-(1H-Benzimidazol-1-yl)-9-[1 (R)-(3-pyridyl)ethyl]-8,9-dihydro-7H-purin-8-one; 2-(1H-Benzimidazol-1-yl)-9-[4-oxo-1,2,3,4-tetrahydronaphthalen-1(R)-yl]-8,9-dihydro-7H-purin-8-one; 1-[9-[6-Fluoro-3,4-dihydro-2H-1-benzopyran-4(R)-yl]-8-oxo-8,9-dihydro-7H-purin-2-yl]-1H-benzimidazole-6-carbonitrile; 1-[9-[7-Fluoro-3,4-dihydro-2H-1-benzopyran-4(R)-yl]-8-oxo-8,9-dihydro-7H-purin-2-yl]-1H-benzimidazole-6-carbonitrile; and 2-(1H-Benzimidazol-1-yl)-9-[5,8-difluoro-3,4-dihydro-2H-1-benzopyran-4(R)-yl]-8,9-dihydro-7H-purin-8-one)

Examples of p38 Kinase Inhibitors include, but are not limited to, 3 (5)-heteroaryl substituted pyrazoles (see, U.S. Pat. No. 5,932,425). Additional p38 kinase inhibitors include, but are not limited to, 1-(5-tert-butyl-2-p-tolyl-2H-pyrazol-3-yl)-3-[4-(2-morpholin-4-yl-ethoxy) naphthalen-1-yl]urea (BIRB 796); SB202190; SB203580; VX-745; and VX-702.

Examples of therapeutic agents that treats or inhibits the respiratory disorder that are suitable for use in treating AERD include, but are not limited to: high-dose aspirin therapy, P2Y12 inhibitors, leukotriene receptor antagonists, thromboxane receptor antagonists, and 5-lipoxygenase inhibitors.

Examples of P2Y12 inhibitors include, but are not limited to, PLAVIX™ (clopidogrel), cangrelor, ticagrelor, TICLID™ (ticlopidine), EFFIENT™ (prasugrel), and elinogrel (PRT060128 and PRT128).

Examples of leukotriene receptor antagonists include, but are not limited to, SINGULAIR™ (montelukast) and ACCOLATE™ (zafirlukast).

Examples of thromboxane receptor antagonists include, but are not limited to, HEPATOREN™ (iretroban), SERATRODAST™ (AA-2414), S18886 (terutroban), PTA2, 13-APA, GR-32191, BM-13177 (sulotroban), SQ-29,548, SQ-28,668, ONO-3708, Bay U3405, EP-045, BMS-180,291, S-145, I-BOP ([1S-[1alpha, 2alpha (Z),3beta (1E,3S*),4alpha]]-7-[3-[3-hydroxy-4-(4-iodophenoxy)-1-butenyl]-7-oxabi-cyclo[2.2.1]hept-2-yl]5-heptenoic acid), U46619 (9,11-dideoxy-9alpha11alpha-methanoepoxy-prosta-5Z,13E-dien-1-oic acid), PBT-3 [10 (S)-hydroxy-11,12-cyclopropyl-eicosa-5Z,8Z,14Z-trienoic acid methyl ester], hepoxilin cyclopropane, BM-531 (N-tert-butyl-N′-[(2-cyclohexylamino-5-nitrobenzene) sulfonyl]urea), EV-077, L0655, and ICI 192,605.

Examples of 5-lipoxygenase inhibitors include, but are not limited to, ASTELIN™ and ASTELPRO™ (azelastine), diethylcarbamazine, nordihydroguaiaretic acid, and ZYFLO™ (zileuton).

In some embodiments, the dose of the therapeutic agents that treat or inhibit a respiratory disorder can be reduced by about 10%, by about 20%, by about 30%, by about 40%, by about 50%, by about 60%, by about 70%, by about 80%, or by about 90% for patients or human subjects that are heterozygous for an ALOX15 predicted loss-of-function variant (i.e., a lower than the standard dosage amount) compared to patients or human subjects that are ALOX15 reference (who may receive a standard dosage amount). In some embodiments, the dose of the therapeutic agents that treat or inhibit a respiratory disorder can be reduced by about 10%, by about 20%, by about 30%, by about 40%, or by about 50%. In addition, the dose of therapeutic agents that treat or inhibit a respiratory disorder in patients or human subjects that are heterozygous for an ALOX15 predicted loss-of-function variant can be administered less frequently compared to patients or human subjects that are ALOX15 reference.

Administration of the therapeutic agents that treat or inhibit a respiratory disorder and/or ALOX15 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a patient can receive therapy for a prolonged period of time such as, for example, 6 months, 1 year, or more.

Administration of the therapeutic agents that treat or inhibit a respiratory disorder and/or ALOX15 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in a respiratory disorder, a decrease/reduction in the severity of a respiratory disorder (such as, for example, a reduction or inhibition of development of a respiratory disorder), a decrease/reduction in symptoms and respiratory disorder-related effects, delaying the onset of symptoms and respiratory disorder-related effects, reducing the severity of symptoms of respiratory disorder-related effects, reducing the severity of an acute episode, reducing the number of symptoms and respiratory disorder-related effects, reducing the latency of symptoms and respiratory disorder-related effects, an amelioration of symptoms and respiratory disorder-related effects, reducing secondary symptoms, reducing secondary infections, preventing relapse to a respiratory disorder, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of respiratory disorder development/progression (such as, for example, a complete or partial avoidance/inhibition or a delay), and an increased survival time of the affected host animal, following administration of a therapeutic protocol. Treatment of a respiratory disorder encompasses the treatment of patients already diagnosed as having any form of a respiratory disorder at any clinical stage or manifestation, the delay of the onset or evolution or aggravation or deterioration of the symptoms or signs of a respiratory disorder, and/or preventing and/or reducing the severity of a respiratory disorder.

The present disclosure also provides methods of identifying a human subject having an increased risk for developing a respiratory disorder. In some embodiments, the method comprises determining or having determined in a biological sample obtained from the subject the presence or absence of an ALOX15 predicted loss-of-function variant nucleic acid molecule (such as a genomic nucleic acid molecule, mRNA molecule, and/or cDNA molecule) encoding a human ALOX15 polypeptide. When the human subject lacks an ALOX15 predicted loss-of-function variant nucleic acid molecule (i.e., the human subject is genotypically categorized as an ALOX15 reference), then the human subject has an increased risk for developing a respiratory disorder. When the human subject has an ALOX15 predicted loss-of-function variant nucleic acid molecule (i.e., the human subject is heterozygous for an ALOX15 predicted loss-of-function variant or homozygous for an ALOX15 predicted loss-of-function variant), then the human subject has a decreased risk for developing a respiratory disorder.

Having a single copy of an ALOX15 predicted loss-of-function variant nucleic acid molecule is more protective of a human subject from developing a respiratory disorder than having no copies of an ALOX15 predicted loss-of-function variant nucleic acid molecule. Without intending to be limited to any particular theory or mechanism of action, it is believed that a single copy of an ALOX15 predicted loss-of-function variant nucleic acid molecule (i.e., heterozygous for an ALOX15 predicted loss-of-function variant) is protective of a human subject from developing a respiratory disorder, and it is also believed that having two copies of an ALOX15 predicted loss-of-function variant nucleic acid molecule (i.e., homozygous for an ALOX15 predicted loss-of-function variant) may be more protective of a human subject from developing a respiratory disorder, relative to a human subject with a single copy. Thus, in some embodiments, a single copy of an ALOX15 predicted loss-of-function variant nucleic acid molecule may not be completely protective, but instead, may be partially or incompletely protective of a human subject from developing a respiratory disorder. While not desiring to be bound by any particular theory, there may be additional factors or molecules involved in the development of a respiratory disorder that are still present in a human subject having a single copy of an ALOX15 predicted loss-of-function variant nucleic acid molecule, thus resulting in less than complete protection from the development of a respiratory disorder.

Determining whether a human subject has an ALOX15 predicted loss-of-function variant nucleic acid molecule in a biological sample from a patient and/or determining whether a patient has an ALOX15 predicted loss-of-function variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the human subject.

In some embodiments, when a human subject is identified as having an increased risk of developing a respiratory disorder, the human subject is further treated with a therapeutic agent that treats or inhibits a respiratory disorder and/or an ALOX15 inhibitor, as described herein. For example, when the human subject is ALOX15 reference, and therefore has an increased risk for developing a respiratory disorder, the human subject is administered an ALOX15 inhibitor. In some embodiments, such a patient is also administered a therapeutic agent that treats or inhibits a respiratory disorder. In some embodiments, when the patient is heterozygous for an ALOX15 predicted loss-of-function variant, the patient is administered the therapeutic agent that treats or inhibits a respiratory disorder in a dosage amount that is the same as or lower than a standard dosage amount, and is also administered an ALOX15 inhibitor. In some embodiments, the patient is ALOX15 reference. In some embodiments, the patient is heterozygous for an ALOX15 predicted loss-of-function variant.

The present disclosure also provides methods of detecting the presence or absence of an ALOX15 predicted loss-of-function variant genomic nucleic acid molecule in a biological sample from a human subject, and/or an ALOX15 predicted loss-of-function variant mRNA molecule in a biological sample from a human subject, and/or an ALOX15 predicted loss-of-function variant cDNA molecule produced from an mRNA molecule in a biological sample from a human subject. It is understood that gene sequences within a population and mRNA molecules encoded by such genes can vary due to polymorphisms such as single-nucleotide polymorphisms. The sequences provided herein for the ALOX15 variant genomic nucleic acid molecule, ALOX15 variant mRNA molecule, and ALOX15 variant cDNA molecule are only exemplary sequences. Other sequences for the ALOX15 variant genomic nucleic acid molecule, variant mRNA molecule, and variant cDNA molecule are also possible.

The biological sample can be derived from any cell, tissue, or biological fluid from the subject. The sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The sample used in the methods disclosed herein will vary based on the assay format, nature of the detection method, and the tissues, cells, or extracts that are used as the sample. A biological sample can be processed differently depending on the assay being employed. For example, when detecting any ALOX15 variant nucleic acid molecule, preliminary processing designed to isolate or enrich the sample for the genomic DNA can be employed. A variety of techniques may be used for this purpose. When detecting the level of any ALOX15 variant mRNA, different techniques can be used enrich the biological sample with mRNA. Various methods to detect the presence or level of an mRNA or the presence of a particular variant genomic DNA locus can be used.

In some embodiments, detecting a human ALOX15 predicted loss-of-function variant nucleic acid molecule in a human subject comprises assaying or genotyping a biological sample obtained from the human subject to determine whether an ALOX15 genomic nucleic acid molecule in the biological sample, and/or an ALOX15 mRNA molecule in the biological sample, and/or an ALOX15 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an ALOX15 predicted loss-of-function variant nucleic acid molecule (such as, for example, a genomic nucleic acid molecule, an mRNA molecule, and/or a cDNA molecule produced from an mRNA molecule) in a human subject, comprise performing an assay on a biological sample obtained from the human subject. The assay determines whether a nucleic acid molecule in the biological sample comprises a particular nucleotide sequence.

In some embodiments, the nucleotide sequence comprises: a thymine at a position corresponding to position 9,917 according to SEQ ID NO:2 (for genomic nucleic acid molecules); a uracil at a position corresponding to position 1,693 according to SEQ ID NO:4 (for mRNA molecules); or a thymine at a position corresponding to position 1,693 according to SEQ ID NO:6 (for cDNA molecules obtained from mRNA molecules). In some embodiments, the nucleotide sequence comprises a thymine at a position corresponding to position 9,917 according to SEQ ID NO:2, or the complement thereof. In some embodiments, the nucleotide sequence comprises a uracil at a position corresponding to position 1,693 according to SEQ ID NO:4, or the complement thereof. In some embodiments, the nucleotide sequence comprises a thymine at a position corresponding to position 1,693 according to SEQ ID NO:6, or the complement thereof.

In some embodiments, the biological sample comprises a cell or cell lysate. Such methods can further comprise, for example, obtaining a biological sample from the subject comprising an ALOX15 genomic nucleic acid molecule or mRNA molecule, and if mRNA, optionally reverse transcribing the mRNA into cDNA. Such assays can comprise, for example determining the identity of these positions of the particular ALOX15 nucleic acid molecule. In some embodiments, the method is an in vitro method.

In some embodiments, the determining step, detecting step, or genotyping assay comprises sequencing at least a portion of the nucleotide sequence of the ALOX15 genomic nucleic acid molecule, the ALOX15 mRNA molecule, or the ALOX15 cDNA molecule in the biological sample, wherein the sequenced portion comprises one or more variations that cause a loss-of-function (partial or complete) or are predicted to cause a loss-of-function (partial or complete).

In some embodiments, the determining step, detecting step, or genotyping assay comprises sequencing at least a portion of: the nucleotide sequence of the ALOX15 genomic nucleic acid molecule in the biological sample, wherein the sequenced portion comprises a position corresponding to position 9,917 according to SEQ ID NO:2, or the complement thereof; the nucleotide sequence of the ALOX15 mRNA molecule in the biological sample, wherein the sequenced portion comprises a position corresponding to position 1,693 according to SEQ ID NO:4, or the complement thereof; and/or the nucleotide sequence of the ALOX15 cDNA molecule produced from the mRNA in the biological sample, wherein the sequenced portion comprises a position corresponding to position 1,693 according to SEQ ID NO: 6, or the complement thereof. When the sequenced portion of the ALOX15 nucleic acid molecule in the biological sample comprises a thymine at a position corresponding to position 9,917 according to SEQ ID NO:2, a uracil at a position corresponding to position 1,693 according to SEQ ID NO:4, or a thymine at a position corresponding to position 1,693 according to SEQ ID NO:6, then the ALOX15 nucleic acid molecule in the biological sample is an ALOX15 predicted loss-of-function variant nucleic acid molecule.

In some embodiments, the determining step, detecting step, or genotyping assay comprises sequencing at least a portion of the nucleotide sequence of the ALOX15 genomic nucleic acid molecule in the biological sample, wherein the sequenced portion comprises a position corresponding to position 9,917 according to SEQ ID NO:2, or the complement thereof. When the sequenced portion of the ALOX15 nucleic acid molecule in the biological sample comprises a thymine at a position corresponding to position 9,917 according to SEQ ID NO: 2, then the ALOX15 nucleic acid molecule in the biological sample is an ALOX15 predicted loss-of-function variant nucleic acid molecule.

In some embodiments, the determining step, detecting step, or genotyping assay comprises sequencing at least a portion of the nucleotide sequence of the ALOX15 mRNA molecule in the biological sample, wherein the sequenced portion comprises a position corresponding to position 1,693 according to SEQ ID NO:4, or the complement thereof. When the sequenced portion of the ALOX15 nucleic acid molecule in the biological sample comprises a uracil at a position corresponding to position 1,693 according to SEQ ID NO:4, then the ALOX15 nucleic acid molecule in the biological sample is an ALOX15 predicted loss-of-function variant nucleic acid molecule.

In some embodiments, the determining step, detecting step, or genotyping assay comprises sequencing at least a portion of the nucleotide sequence of the ALOX15 cDNA molecule in the biological sample, wherein the sequenced portion comprises a position corresponding to position 1,693 according to SEQ ID NO:6, or the complement thereof. When the sequenced portion of the ALOX15 nucleic acid molecule in the biological sample comprises a thymine at a position corresponding to position 1,693 according to SEQ ID NO: 6, then the ALOX15 nucleic acid molecule in the biological sample is an ALOX15 predicted loss-of-function variant nucleic acid molecule.

In some embodiments, the determining step, detecting step, or genotyping assay comprises: a) contacting the biological sample with a primer hybridizing to a portion of the nucleotide sequence of the ALOX15: genomic nucleic acid molecule that is proximate to a position corresponding to position 9,917 according to SEQ ID NO:2; mRNA molecule that is proximate to a position corresponding to position 1,693 according to SEQ ID NO:4; and/or cDNA molecule that is proximate to a position corresponding to position 1,693 according to SEQ ID NO: 6; b) extending the primer at least through the position of the nucleotide sequence of the ALOX15: genomic nucleic acid molecule corresponding to position 9,917 according to SEQ ID NO:2; mRNA molecule corresponding to position 1,693 according to SEQ ID NO:4; and/or cDNA molecule corresponding to position 1,693 according to SEQ ID NO: 6; and c) determining whether the extension product of the primer comprises: a thymine at a position corresponding to position 9,917 according to SEQ ID NO:2; a uracil at a position corresponding to position 1,693 according to SEQ ID NO:4; and/or a thymine at a position corresponding to position 1,693 according to SEQ ID NO:6.

In some embodiments, the determining step, detecting step, or genotyping assay comprises: a) contacting the biological sample with a primer hybridizing to a portion of the nucleotide sequence of the ALOX15 genomic nucleic acid molecule that is proximate to a position corresponding to position 9,917 according to SEQ ID NO:2; b) extending the primer at least through the position of the nucleotide sequence of the ALOX15 genomic nucleic acid molecule corresponding to position 9,917 according to SEQ ID NO:2; and c) determining whether the extension product of the primer comprises a thymine at a position corresponding to position 9,917 according to SEQ ID NO:2.

In some embodiments, the determining step, detecting step, or genotyping assay comprises: a) contacting the biological sample with a primer hybridizing to a portion of the nucleotide sequence of the ALOX15 mRNA molecule that is proximate to a position corresponding to position 1,693 according to SEQ ID NO:4; b) extending the primer at least through the position of the nucleotide sequence of the ALOX15 mRNA molecule corresponding to position 1,693 according to SEQ ID NO:4; and c) determining whether the extension product of the primer comprises a uracil at a position corresponding to position 1,693 according to SEQ ID NO:4.

In some embodiments, the determining step, detecting step, or genotyping assay comprises: a) contacting the biological sample with a primer hybridizing to a portion of the nucleotide sequence of the ALOX15 cDNA molecule that is proximate to a position corresponding to position 1,693 according to SEQ ID NO:6; b) extending the primer at least through the position of the nucleotide sequence of the ALOX15 cDNA molecule corresponding to position 1,693 according to SEQ ID NO:6; and c) determining whether the extension product of the primer comprises a thymine at a position corresponding to position 1,693 according to SEQ ID NO:6.

In some embodiments, the assay comprises sequencing the entire nucleic acid molecule. In some embodiments, only an ALOX15 genomic nucleic acid molecule is analyzed. In some embodiments, only an ALOX15 mRNA is analyzed. In some embodiments, only an ALOX15 cDNA obtained from ALOX15 mRNA is analyzed.

In some embodiments, the determining step, detecting step, or genotyping assay comprises: a) amplifying at least a portion of the nucleic acid molecule that encodes the human ALOX15 polypeptide, wherein the amplified portion comprises: i) a thymine at a position corresponding to position 9,917 according to SEQ ID NO:2, or the complement thereof; ii) a uracil at a position corresponding to position 1,693 according to SEQ ID NO:4, or the complement thereof; and/or iii) a thymine at a position corresponding to position 1,693 according to SEQ ID NO:6, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising: i) a thymine at a position corresponding to position 9,917 according to SEQ ID NO:2, or the complement thereof; ii) a uracil at a position corresponding to position 1,693 according to SEQ ID NO:4, or the complement thereof; and/or iii) a thymine at a position corresponding to position 1,693 according to SEQ ID NO:6, or the complement thereof; and d) detecting the detectable label.

In some embodiments, the determining step, detecting step, or genotyping assay comprises: a) amplifying at least a portion of the nucleic acid molecule that encodes the human ALOX15 polypeptide, wherein the amplified portion comprises a thymine at a position corresponding to position 9,917 according to SEQ ID NO:2, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 9,917 according to SEQ ID NO:2, or the complement thereof; and d) detecting the detectable label.

In some embodiments, the determining step, detecting step, or genotyping assay comprises: a) amplifying at least a portion of the nucleic acid molecule that encodes the human ALOX15 polypeptide, wherein the amplified portion comprises a uracil at a position corresponding to position 1,693 according to SEQ ID NO:4, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a uracil at a position corresponding to position 1,693 according to SEQ ID NO: 4, or the complement thereof; and d) detecting the detectable label.

In some embodiments, the determining step, detecting step, or genotyping assay comprises: a) amplifying at least a portion of the nucleic acid molecule that encodes the human ALOX15 polypeptide, wherein the amplified portion comprises a thymine at a position corresponding to position 1,693 according to SEQ ID NO:6, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a thymine at a position corresponding to position 1,693 according to SEQ ID NO:6, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising: a thymine at a position corresponding to position 9,917 according to SEQ ID NO:2, or the complement thereof; a uracil at a position corresponding to position 1,693 according to SEQ ID NO:4, or the complement thereof; and/or a thymine at a position corresponding to position 1,693 according to SEQ ID NO:6, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 9,917 according to SEQ ID NO:2, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a uracil at a position corresponding to position 1,693 according to SEQ ID NO:4, or the complement thereof; and detecting the detectable label.

In some embodiments, the determining step, detecting step, or genotyping assay comprises: contacting the nucleic acid molecule in the biological sample with an alteration-specific probe comprising a detectable label, wherein the alteration-specific probe comprises a nucleotide sequence which hybridizes under stringent conditions to the nucleotide sequence of the amplified nucleic acid molecule comprising a thymine at a position corresponding to position 1,693 according to SEQ ID NO:6, or the complement thereof; and detecting the detectable label.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the human subject.

In some embodiments, the assay comprises contacting the biological sample with a primer or probe, such as an alteration-specific primer or alteration-specific probe, that specifically hybridizes to an ALOX15 variant genomic sequence, variant mRNA sequence, or variant cDNA sequence and not the corresponding ALOX15 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ALOX15 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an ALOX15 nucleic acid molecule (genomic nucleic acid molecule, mRNA molecule, or cDNA molecule), or complement thereof, within a biological sample comprises a nucleotide sequence comprising a thymine at a position corresponding to position 9,917 according to SEQ ID NO:2 (genomic nucleic acid molecule), or a uracil at a position corresponding to position 1,693 according to SEQ ID NO: 4 (mRNA molecule), or a thymine at a position corresponding to position 1,693 according to SEQ ID NO:6 (cDNA molecule), the biological sample can be subjected to an amplification method using a primer pair that includes a first primer derived from the 5′ flanking sequence adjacent to a thymine at a position corresponding to position 9,917 according to SEQ ID NO:2, or a uracil at a position corresponding to position 1,693 according to SEQ ID NO:4, or a thymine at a position corresponding to position 1,693 according to SEQ ID NO: 6, and a second primer derived from the 3′ flanking sequence adjacent to a thymine at a position corresponding to position 9,917 according to SEQ ID NO:2, or a uracil at a position corresponding to position 1,693 according to SEQ ID NO:4, or a thymine at a position corresponding to position 1,693 according to SEQ ID NO:6 to produce an amplicon that is indicative of the presence of the SNP at positions encoding a thymine at a position corresponding to position 9,917 according to SEQ ID NO:2, or a uracil at a position corresponding to position 1,693 according to SEQ ID NO:4, or a thymine at a position corresponding to position 1,693 according to SEQ ID NO:6. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including positions comprising a thymine at a position corresponding to position 9,917 according to SEQ ID NO:2, or a uracil at a position corresponding to position 1,693 according to SEQ ID NO:4, or a thymine at a position corresponding to position 1,693 according to SEQ ID NO:6, and at least 1, 2, 3, 4, 5, 6, 7, 8, 9, 10, or more nucleotides on each side of positions comprising a thymine at a position corresponding to position 9,917 according to SEQ ID NO:2, or a uracil at a position corresponding to position 1,693 according to SEQ ID NO:4, or a thymine at a position corresponding to position 1,693 according to SEQ ID NO: 6.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of a human ALOX15 predicted loss-of-function polypeptide comprising performing an assay on a sample obtained from a human subject to determine whether an ALOX15 polypeptide in the subject contains one or more variations that causes the polypeptide to have a loss-of-function (partial or complete) or predicted loss-of-function (partial or complete). The ALOX15 predicted loss-of-function polypeptide can be any of the ALOX15 variant polypeptides described herein. In some embodiments, the methods detect the presence of ALOX15 Thr560Met, Tyr139Cys, Leu651fs, Pro565Leu, Asn658Lys, Gly283Arg, Val474Ala, Gly422Arg, or Leu106fs. In some embodiments, the methods detect the presence of ALOX15 Thr560Met.

In some embodiments, the methods comprise performing an assay on a sample obtained from a human subject to determine whether an ALOX15 polypeptide in the sample comprises a methionine at a position corresponding to position 560 according to SEQ ID NO: 8.

In some embodiments, the detecting step comprises sequencing at least a portion of the polypeptide that comprises a position corresponding to position 560 according to SEQ ID NO: 8 or SEQ ID NO:7.

In some embodiments, the detecting step comprises an immunoassay for detecting the presence of a polypeptide that comprises a position corresponding to position 560 according to SEQ ID NO:8 or SEQ ID NO:7.

In some embodiments, when the human subject does not have an ALOX15 predicted loss-of-function polypeptide, then the human subject has an increased risk for developing a respiratory disorder, such as a nasal polyp, allergic rhinitis, asthma, and/or AERD. In some embodiments, when the human subject has an ALOX15 predicted loss-of-function polypeptide, then the human subject has a decreased risk for developing a respiratory disorder, such as a nasal polyp, allergic rhinitis, asthma, and/or AERD, and/or atopy.

The present disclosure also provides isolated nucleic acid molecules that hybridize to ALOX15 variant genomic nucleic acid molecules, ALOX15 variant mRNA molecules, and/or ALOX15 variant cDNA molecules (such as any of the genomic variant nucleic acid molecules, mRNA variant molecules, and cDNA variant molecules disclosed herein). In some embodiments, the isolated nucleic acid molecules hybridize to a portion of the ALOX15 nucleic acid molecule that includes a position corresponding to position 9,917 according to SEQ ID NO:2, position 1,693 according to SEQ ID NO:4, or position 1,693 according to SEQ ID NO: 6.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to ALOX15 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ALOX15 variant genomic nucleic acid molecules, ALOX15 variant mRNA molecules, and/or ALOX15 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to a portion of a nucleotide sequence encoding a human ALOX15 polypeptide, wherein the portion comprises a position corresponding to: position 9,917 according to SEQ ID NO:2, or the complement thereof; position 1,693 according to SEQ ID NO:4, or the complement thereof; or position 1,693 according to SEQ ID NO:6, or the complement thereof. In some embodiments, the alteration-specific probe or alteration-specific primer comprises a nucleotide sequence which is complementary to a portion of a nucleotide sequence comprising positions corresponding to: positions 9,916 to 9,918 according to SEQ ID NO:2, or the complement thereof; positions 1,692 to 1,694 according to SEQ ID NO:4, or the complement thereof; and/or positions 1,692 to 1,694 according to SEQ ID NO:6, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ALOX15 variant genomic nucleic acid molecules, ALOX15 variant mRNA molecules, and/or ALOX15 variant cDNA molecules disclosed herein. The primers described herein can be used to amplify ALOX15 variant genomic nucleic acid molecules, ALOX15 variant mRNA molecules, or ALOX15 variant cDNA molecules, or a fragment thereof.

The present disclosure also provides pairs of primers comprising any of the primers described above. For example, if one of the primers' 3′-ends hybridizes to a cytosine at a position corresponding to position 9,917 according to SEQ ID NO:1 (rather than thymine) in a particular ALOX15 nucleic acid molecule, then the presence of the amplified fragment would indicate the presence of an ALOX15 reference genomic nucleic acid molecule. Conversely, if one of the primers' 3′-ends hybridizes to a thymine at a position corresponding to position 9,917 according to SEQ ID NO:2 (rather than cytosine) in a particular ALOX15 nucleic acid molecule, then the presence of the amplified fragment would indicate the presence of the ALOX15 variant genomic nucleic acid molecule. In some embodiments, the nucleotide of the primer complementary to the thymine at a position corresponding to position 9,917 according to SEQ ID NO:2 can be at the 3′ end of the primer. In addition, if one of the primers' 3′-ends hybridizes to a cytosine at a position corresponding to position 1,693 according to SEQ ID NO:3 (rather than uracil) in a particular ALOX15 nucleic acid molecule, then the presence of the amplified fragment would indicate the presence of an ALOX15 reference mRNA molecule. Conversely, if one of the primers' 3′-ends hybridizes to a uracil at a position corresponding to position 1,693 according to SEQ ID NO:4 (rather than cytosine) in a particular ALOX15 mRNA molecule, then the presence of the amplified fragment would indicate the presence of the ALOX15 variant mRNA molecule. In some embodiments, the nucleotide of the primer complementary to the uracil at a position corresponding to position 1,693 according to SEQ ID NO:4 can be at the 3′ end of the primer. In addition, if one of the primers' 3′-ends hybridizes to a cytosine at a position corresponding to position 1,693 according to SEQ ID NO:5 (rather than thymine) in a particular ALOX15 nucleic acid molecule, then the presence of the amplified fragment would indicate the presence of an ALOX15 reference cDNA molecule. Conversely, if one of the primers' 3′-ends hybridizes to a thymine at a position corresponding to position 1,693 according to SEQ ID NO:6 (rather than cytosine) in a particular ALOX15 cDNA molecule, then the presence of the amplified fragment would indicate the presence of the ALOX15 variant cDNA molecule. In some embodiments, the nucleotide of the primer complementary to the thymine at a position corresponding to position 1,693 according to SEQ ID NO:6 can be at the 3′ end of the primer.

In the context of the disclosure “specifically hybridizes” means that the probe or primer (such as, for example, the alteration-specific probe or alteration-specific primer) does not hybridize to a nucleic acid sequence encoding an ALOX15 reference genomic nucleic acid molecule, an ALOX15 reference mRNA molecule, and/or an ALOX15 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The present disclosure also provides molecular complexes comprising or consisting of any of the ALOX15 nucleic acid molecules (genomic nucleic acid molecules, mRNA molecules, or cDNA molecules), or complement thereof, described herein and any of the alteration-specific primers or alteration-specific probes described herein. In some embodiments, the ALOX15 nucleic acid molecules (genomic nucleic acid molecules, mRNA molecules, or cDNA molecules), or complement thereof, in the molecular complexes are single-stranded. In some embodiments, the ALOX15 nucleic acid molecule is any of the genomic nucleic acid molecules described herein. In some embodiments, the ALOX15 nucleic acid molecule is any of the mRNA molecules described herein. In some embodiments, the ALOX15 nucleic acid molecule is any of the cDNA molecules described herein. In some embodiments, the molecular complex comprises or consists of any of the ALOX15 nucleic acid molecules (genomic nucleic acid molecules, mRNA molecules, or cDNA molecules), or complement thereof, described herein and any of the alteration-specific primers described herein. In some embodiments, the molecular complex comprises or consists of any of the ALOX15 nucleic acid molecules (genomic nucleic acid molecules, mRNA molecules, or cDNA molecules), or complement thereof, described herein and any of the alteration-specific probes described herein.

In some embodiments, the molecular complex comprises or consists of an alteration-specific primer or an alteration-specific probe hybridized to a genomic nucleic acid molecule comprising a nucleotide sequence encoding a human ALOX15 polypeptide, wherein the alteration-specific primer or the alteration-specific probe is hybridized to a thymine at a position corresponding to position 9,917 according to SEQ ID NO:2, or the complement thereof.

In some embodiments, the molecular complex comprises or consists of an alteration-specific primer or an alteration-specific probe that is hybridized to an ATG codon at positions corresponding to positions 9,916 to 9,918 according to SEQ ID NO:2.

In some embodiments, the molecular complex comprises or consists of a genomic nucleic acid molecule that comprises SEQ ID NO:2.

In some embodiments, the molecular complex comprises or consists of an alteration-specific primer or an alteration-specific probe hybridized to an mRNA molecule comprising a nucleotide sequence encoding a human ALOX15 polypeptide, wherein the alteration-specific primer or the alteration-specific probe is hybridized to a uracil at a position corresponding to position 1,693 according to SEQ ID NO:4, or the complement thereof.

In some embodiments, the molecular complex comprises or consists of an alteration-specific primer or an alteration-specific probe that is hybridized to an AUG codon at positions corresponding to positions 1,692 to 1,694 according to SEQ ID NO:4.

In some embodiments, the molecular complex comprises or consists of an mRNA molecule that comprises SEQ ID NO:4.

In some embodiments, the molecular complex comprises or consists of an alteration-specific primer or an alteration-specific probe hybridized to a cDNA molecule comprising a nucleotide sequence encoding a human ALOX15 polypeptide, wherein the alteration-specific primer or the alteration-specific probe is hybridized to a thymine at a position corresponding to position 1,693 according to SEQ ID NO:6, or the complement thereof.

In some embodiments, the molecular complex comprises or consists of an alteration-specific primer or an alteration-specific probe that is hybridized to an ATG codon at positions corresponding to positions 1,692 to 1,694 according to SEQ ID NO:6.

In some embodiments, the molecular complex comprises or consists of a cDNA molecule that comprises SEQ ID NO:6.

In some embodiments, the molecular complex comprises an alteration-specific probe or an alteration-specific primer comprising a label. In some embodiments, the label is a fluorescent label, a radiolabel, or biotin. In some embodiments, the molecular complex further comprises a non-human polymerase.

The nucleotide sequence of an ALOX15 reference genomic nucleic acid molecule is set forth in SEQ ID NO:1. Referring to SEQ ID NO:1, position 9,917 is a cytosine.

A variant genomic nucleic acid molecule of ALOX15 exists, wherein the cytosine at position 9,917 is replaced with thymine. The nucleotide sequence of this ALOX15 variant genomic nucleic acid molecule is set forth in SEQ ID NO:2.

The nucleotide sequence of an ALOX15 reference mRNA molecule is set forth in SEQ ID NO:3. Referring to SEQ ID NO:3, position 1,693 is a cytosine.

A variant mRNA molecule of ALOX15 exists, wherein the cytosine at position 1,693 is replaced with uracil. The nucleotide sequence of this ALOX15 variant mRNA molecule is set forth in SEQ ID NO:4.

The nucleotide sequence of an ALOX15 reference cDNA molecule is set forth in SEQ ID NO: 5. Referring to SEQ ID NO:5, position 1,693 is a cytosine.

A variant cDNA molecule of ALOX15 exists, wherein the cytosine at position 1,693 is replaced with thymine. The nucleotide sequence of this ALOX15 variant cDNA molecule is set forth in SEQ ID NO:6.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides compositions comprising any one or more of the isolated nucleic acid molecules, genomic nucleic acid molecules, mRNA molecules, and/or cDNA molecules disclosed herein. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:3, or SEQ ID NO:5). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

For example, a nucleic acid molecule comprising a nucleotide sequence encoding a human ALOX15 polypeptide, wherein the nucleotide sequence comprises a thymine at a position corresponding to position 9,917 according to SEQ ID NO:2 means that if the nucleotide sequence of the ALOX15 genomic nucleic acid molecule is aligned to the sequence of SEQ ID NO:2, the ALOX15 sequence has a thymine residue at the position that corresponds to position 9,917 of SEQ ID NO:2. The same applies for mRNA molecules comprising a nucleotide sequence encoding a human ALOX15 polypeptide, wherein the nucleotide sequence comprises a uracil at a position corresponding to position 1,693 according to SEQ ID NO:4, and cDNA molecules comprising a nucleotide sequence encoding a human ALOX15 polypeptide, wherein the nucleotide sequence comprises a thymine at a position corresponding to position 1,693 according to SEQ ID NO:6. In other words, these phrases refer to a nucleic acid molecule encoding an ALOX15 polypeptide, wherein the genomic nucleic acid molecule has a nucleotide sequence that comprises a thymine residue that is homologous to the thymine residue at position 9,917 of SEQ ID NO:2 (or wherein the mRNA molecule has a nucleotide sequence that comprises a uracil residue that is homologous to the uracil residue at position 1,693 of SEQ ID NO:4, or wherein the cDNA molecule has a nucleotide sequence that comprises a thymine residue that is homologous to the thymine residue at position 1,693 of SEQ ID NO:6). Herein, such a sequence is also referred to as “ALOX15 sequence with the C9,917T alteration” or “ALOX15 sequence with the C9,917T variation” referring to genomic nucleic acid molecules (or “ALOX15 sequence with the C1,693U alteration” or “ALOX15 sequence with the C1,693U variation” referring to mRNA molecules, and “ALOX15 sequence with the C1,693T alteration” or “ALOX15 sequence with the C1,693T variation” referring to cDNA molecules).

As described herein, a position within an ALOX15 genomic nucleic acid molecule that corresponds to position 9,917 according to SEQ ID NO:2, for example, can be identified by performing a sequence alignment between the nucleotide sequence of a particular ALOX15 nucleic acid molecule and the nucleotide sequence of SEQ ID NO:2. A variety of computational algorithms exist that can be used for performing a sequence alignment to identify a nucleotide position that corresponds to, for example, position 9,917 in SEQ ID NO: 2. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequence of an ALOX15 reference polypeptide is set forth in SEQ ID NO: 7. Referring to SEQ ID NO:7, the ALOX15 reference polypeptide is 662 amino acids in length. Referring to SEQ ID NO:7, position 560 is threonine.

An ALOX15 variant polypeptide exists (Thr560Met or T560M), the amino acid sequence of which is set forth in SEQ ID NO:8. Referring to SEQ ID NO:8, the ALOX15 variant polypeptide is 662 amino acids in length. Referring to SEQ ID NO:8, position 560 is methionine.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or inhibit a respiratory disorder for use in the treatment of a respiratory disorder (or for use in the preparation of a medicament for treating a respiratory disorder) in a human subject, wherein the human subject has any of the genomic nucleic acid molecules, mRNA molecules, and/or cDNA molecules encoding a human ALOX15 polypeptide described herein. The therapeutic agents that treat or inhibit a respiratory disorder can be any of the therapeutic agents that treat or inhibit a respiratory disorder described herein.

In some embodiments, the human subject comprises: a genomic nucleic acid molecule having a nucleotide sequence encoding a human ALOX15 polypeptide, wherein the nucleotide sequence comprises a thymine at a position corresponding to position 9,917 according to SEQ ID NO:2, or the complement thereof; an mRNA molecule having a nucleotide sequence encoding a human ALOX15 polypeptide, wherein the nucleotide sequence comprises a uracil at a position corresponding to position 1,693 according to SEQ ID NO: 4, or the complement thereof; a cDNA molecule having a nucleotide sequence encoding a human ALOX15 polypeptide, wherein the nucleotide sequence comprises a thymine at a position corresponding to position 1,693 according to SEQ ID NO:6, or the complement thereof; or an ALOX15 polypeptide that comprises a methionine at a position corresponding to position 560 according to SEQ ID NO:8.

The present disclosure also provides ALOX15 inhibitors for use in the treatment of a respiratory disorder (or for use in the preparation of a medicament for treating a respiratory disorder) in a human subject, wherein the human subject has any of the genomic nucleic acid molecules, mRNA molecules, and/or cDNA molecules encoding a human ALOX15 polypeptide described herein. The ALOX15 inhibitors can be any of the ALOX15 inhibitors described herein.

In some embodiments, the human subject comprises: a genomic nucleic acid molecule having a nucleotide sequence encoding a human ALOX15 polypeptide, wherein the nucleotide sequence comprises a thymine at a position corresponding to position 9,917 according to SEQ ID NO:2, or the complement thereof; an mRNA molecule having a nucleotide sequence encoding a human ALOX15 polypeptide, wherein the nucleotide sequence comprises a uracil at a position corresponding to position 1,693 according to SEQ ID NO: 4, or the complement thereof; a cDNA molecule having a nucleotide sequence encoding a human ALOX15 polypeptide, wherein the nucleotide sequence comprises a thymine at a position corresponding to position 1,693 according to SEQ ID NO:6, or the complement thereof; or an ALOX15 polypeptide that comprises a methionine at a position corresponding to position 560 according to SEQ ID NO:8. The ALOX15 inhibitors can be any of the ALOX15 inhibitors described herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Exome Sequencing Analysis

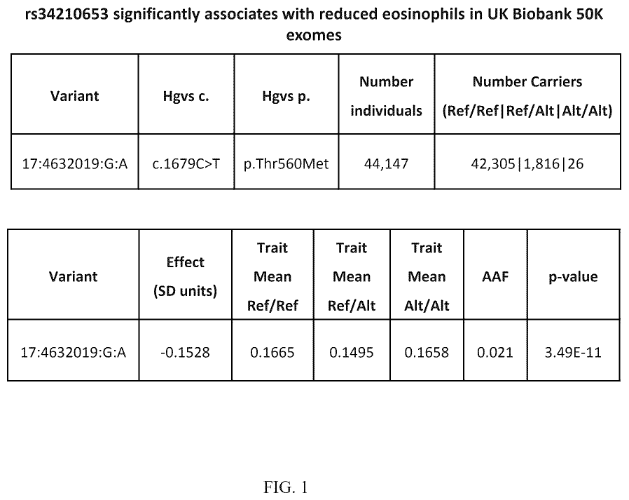

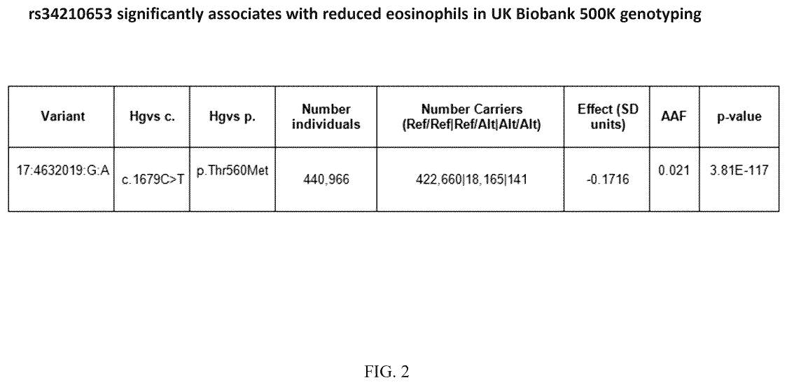

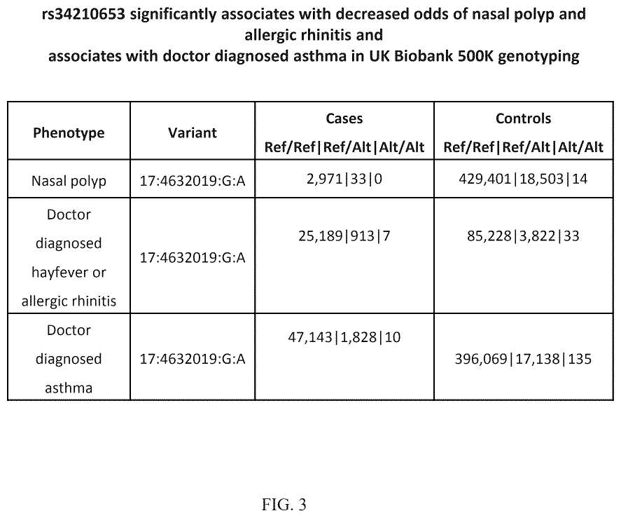

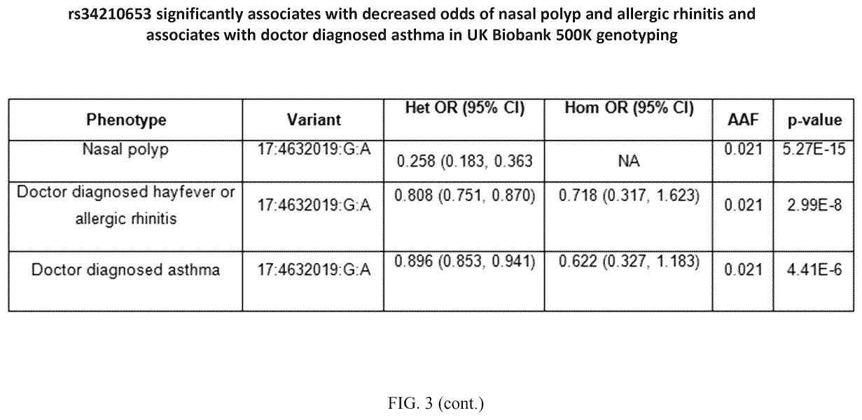

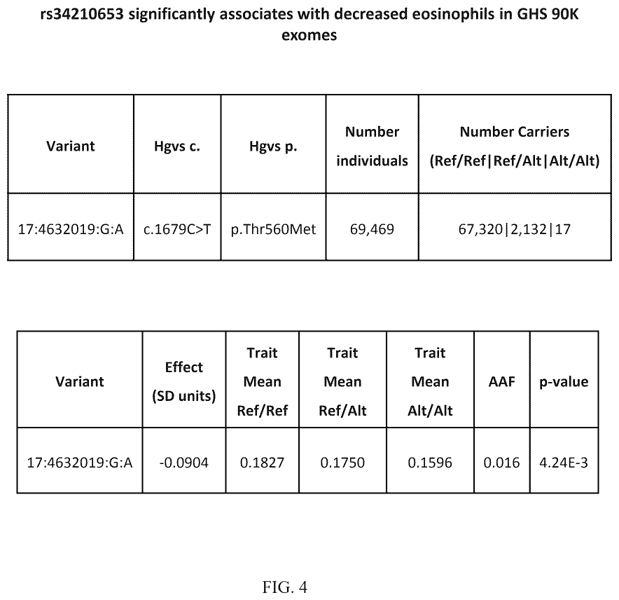

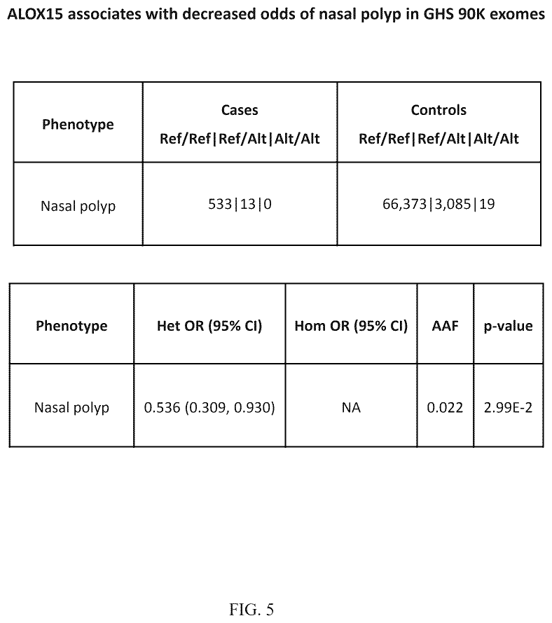

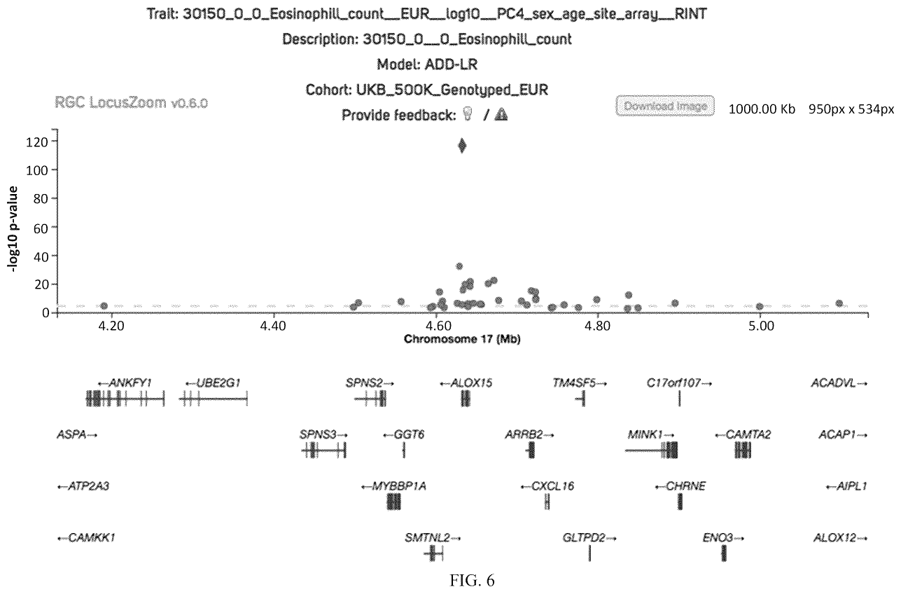

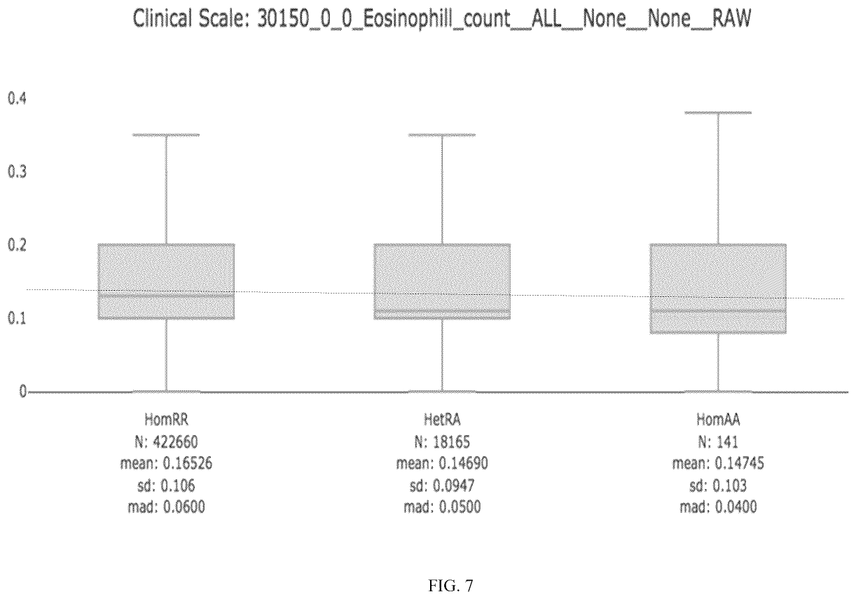

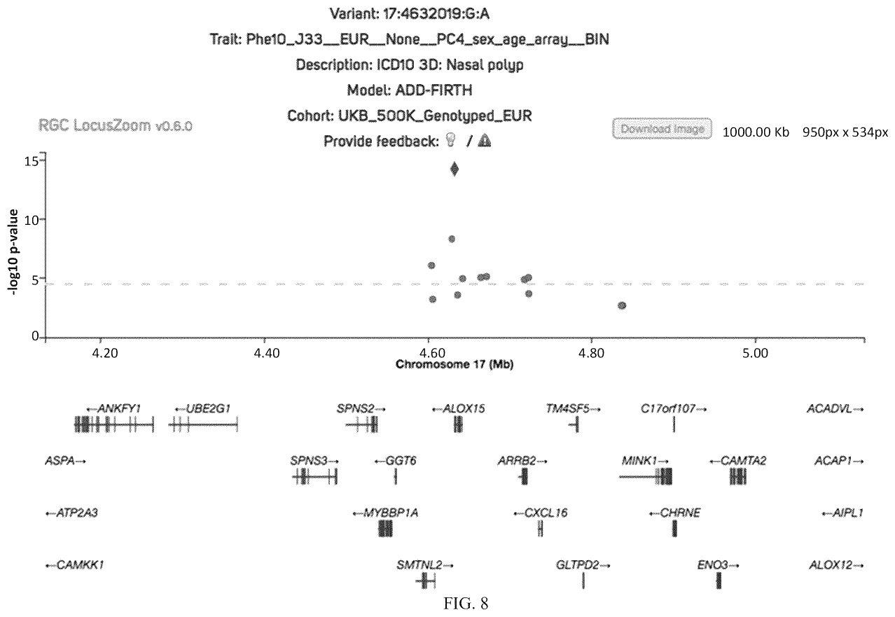

Exome sequencing and analysis at the Regeneron Genetics Center identified that rs34210653 significantly associates with decreased circulating blood eosinophils in UK Biobank 50K exome data (see, FIG. 1) and UK Biobank 500K genotyping data (see, FIG. 2) and also associates with decreased circulating blood eosinophils in GHS 90K exome data (see, FIG. 4). The analysis also identified that rs34210653 significantly associates with decreased odds of nasal polyps, allergic rhinitis, and asthma in UK Biobank 500K genotyping data (see, FIG. 3) and with decreased odds of nasal polyps in GHS 90K exomes (see, FIG. 5). A locus zoom plot for the significant association between rs34210653 and eosinophils in UKB 500K genotyped data (see, FIG. 6). Quantitative trait distributions for eosinophil counts in UKB 500K genotyping data showing decreased eosinophils among heterozygous and homozygous rs34210653 variant carriers (dotted line indicates mean eosinophil numbers for wildtype allele carriers) (see, FIG. 7). A locus zoom plot for the significant association between rs34210653 and nasal polyps in UKB 500K genotyped data (see, FIG. 8).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a patient having a nasal polyp, allergic rhinitis, asthma, and/or aspirin-exacerbated respiratory disease (AERD), the method comprising administering an Arachidonate 15-Lipoxygenase inhibitor to the patient.

2-34. (canceled)

35. A method of treating a patient with a therapeutic agent that treats or inhibits a respiratory disorder and an Arachidonate 15-Lipoxygenase (ALOX15) inhibitor, wherein the patient is suffering from a respiratory disorder, the method comprising:
determining whether the patient has an ALOX15 predicted loss-of-function variant nucleic acid molecule encoding a human ALOX15 polypeptide by:
obtaining or having obtained a biological sample from the patient; and
performing or having performed a genotyping assay on the biological sample to determine if the patient has a genotype comprising the ALOX15 predicted loss-of-function variant nucleic acid molecule; and
administering or continuing to administer to the patient the therapeutic agent that treats or inhibits a respiratory disorder in a standard dosage amount, and administering to the patient an ALOX15 inhibitor when the patient is ALOX15 reference; or
administering or continuing to administer to the patient the therapeutic agent that treats or inhibits a respiratory disorder in an amount that is lower than a standard dosage amount, and administering to the patient an ALOX15 inhibitor when the patient is heterozygous for the ALOX15 predicted loss-of-function variant;
wherein the presence of a genotype having the ALOX15 predicted loss-of-function variant nucleic acid molecule encoding the human ALOX15 polypeptide indicates the patient has a reduced risk of developing a respiratory disorder.

36. The method according to claim 35, wherein the patient is ALOX15 reference, and the patient is administered or continued to be administered the therapeutic agent that treats or inhibits a respiratory disorder in a standard dosage amount, and is administered an ALOX15 inhibitor.

37. The method according to claim 35, wherein the patient is heterozygous for the ALOX15 predicted loss-of-function variant, and the patient is administered or continued to be administered the therapeutic agent that treats or inhibits a respiratory disorder in an amount that is lower than a standard dosage amount, and is administered an ALOX15 inhibitor.

38. The method according to claim 35, wherein the ALOX15 predicted loss-of-function variant nucleic acid molecule comprises a nucleic acid molecule encoding ALOX15 Thr560Met, Tyr139Cys, Leu651fs, Pro565Leu, Asn658Lys, Gly283 Arg, Val474Ala, Gly422Arg, or Leu106fs.

39. The method according to claim 35, wherein the ALOX15 predicted loss-of-function variant nucleic acid molecule comprises a nucleic acid molecule encoding ALOX15 Thr560Met.

40. The method according to claim 35, wherein the ALOX15 predicted loss-of-function variant nucleic acid molecule is comprises:
a genomic nucleic acid molecule having a nucleotide sequence comprising a thymine at a position corresponding to position 9,917 according to SEQ ID NO:2;
an mRNA molecule having a nucleotide sequence comprising a uracil at a position corresponding to position 1,693 according to SEQ ID NO:4; or
a cDNA molecule obtained from an mRNA molecule, wherein the cDNA molecule has a nucleotide sequence comprising a thymine at a position corresponding to position 1,693 according to SEQ ID NO:6.

41. The method according to claim 35, wherein the genotyping assay comprises sequencing at least a portion of the nucleotide sequence of the ALOX15 genomic nucleic acid molecule in the biological sample, wherein the sequenced portion comprises a position corresponding to position 9,917 according to SEQ ID NO:2, or the complement thereof; wherein when the sequenced portion of the ALOX15 genomic nucleic acid molecule in the biological sample comprises a thymine at a position corresponding to position 9,917 according to SEQ ID NO:2, then the ALOX15 genomic nucleic acid molecule in the biological sample is an ALOX15 predicted loss-of-function variant genomic nucleic acid molecule.

42. The method according to claim 35, wherein the genotyping assay comprises sequencing at least a portion of the nucleotide sequence of the ALOX15 mRNA molecule in the biological sample, wherein the sequenced portion comprises a position corresponding to position 1,693 according to SEQ ID NO:4, or the complement thereof; wherein when the sequenced portion of the ALOX15 mRNA molecule in the biological sample comprises a uracil at a position corresponding to position 1,693 according to SEQ ID NO:4, then the ALOX15 mRNA molecule in the biological sample is an ALOX15 predicted loss-of-function variant mRNA molecule.

43. The method according to claim 35, wherein the genotyping assay comprises sequencing at least a portion of the nucleotide sequence of the ALOX15 cDNA molecule in the biological sample, wherein the sequenced portion comprises a position corresponding to position 1,693 according to SEQ ID NO:6, or the complement thereof; wherein when the sequenced portion of the ALOX15 cDNA molecule in the biological sample comprises a thymine at a position corresponding to position 1,693 according to SEQ ID NO:6, then the ALOX15 cDNA molecule in the biological sample is an ALOX15 predicted loss-of-function variant cDNA molecule.

44-55. (canceled)

56. The method according to claim 35, wherein the ALOX15 inhibitor comprises an antisense nucleic acid molecule, a small interfering RNA (siRNA), or a short hairpin RNA (shRNA) that hybridizes to an ALOX15 mRNA.

57. The method according to claim 56, wherein the shRNA comprises: a) CCGGGAAACTGGAAGGACGGGTTAACTCGAGTTAACCCGTCCTTCCAGTTTCTTTTT TG (SEQ ID NO:9); b) CCGGGCTATCAAAGACTCTCTAAATCTCGAGATTTAGAGAGTC TTTGATAGCTTTTTG (SEQ ID NO:10); c) CCGGTGGGAAATCATCTATCGGTATCTCGA GATACCGATAGATGATTTCCCATTTTTG (SEQ ID NO:11); d) CCGGCCTGGAAGGAAG ATGCCTTATCTCGAGATAAGGCATCTTCCTTCCAGGTTTTTG (SEQ ID NO:12); e) CCG GCCAGTTTCTTAATGGCGCCAACTCGAGTTGGCGCCATTAAGAAACTGGTTTTTG (SEQ ID NO:13); f) CCGGGCCGTCGATACATCCTATCTTCTCGAGAAGATAGGATGT ATCGACGGCTTTTTG (SEQ ID NO:14); g) CCGGTAGATGACTTCAACCGGATTTCTCG AGAAATCCGGTTGAAGTCATCTATTTTTTG (SEQ ID NO:15); or h) CCGGTGGTACTCT TGGGTGCCTAATCTCGAGATTAGGCACCCAAGAGTACCATTTTTTG (SEQ ID NO:16).

58. The method according to claim 35, wherein the ALOX15 inhibitor comprises a Cas protein and guide RNA (gRNA) that hybridizes to a gRNA recognition sequence within an ALOX15 genomic nucleic acid molecule.

59. The method according to claim 58, wherein the Cas protein is Cas9 or Cpf1.

60. The method according to claim 58, wherein the gRNA recognition sequence includes or is proximate to a position corresponding to: position 9,917 according to SEQ ID NO:1.

61. The method according to claim 58, wherein the gRNA recognition sequence is located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a position corresponding to position 9,917 according to SEQ ID NO:1.

62. The method according to claim 58, wherein a Protospacer Adjacent Motif (PAM) sequence is about 2 to 6 nucleotides downstream of the gRNA recognition sequence.

63. The method according to claim 58, wherein the gRNA comprises from about 17 to about 23 nucleotides.

64. The method according to claim 58, wherein the gRNA recognition sequence comprises a nucleotide sequence according to any one of SEQ ID NOS: 19-43.

65. A method of identifying a human subject having an increased risk for developing a respiratory disorder, wherein the method comprises:
determining or having determined the presence or absence of an ALOX15 predicted loss-of-function variant nucleic acid molecule encoding a human ALOX15 polypeptide in a biological sample obtained from the subject;
wherein:
when the human subject is ALOX15 reference, then the human subject has an increased risk for developing a respiratory disorder; and
when the human subject is heterozygous for an ALOX15 predicted loss-of-function variant or homozygous for an ALOX15 predicted loss-of-function variant, then the human subject has a decreased risk for developing a respiratory disorder.

66-116. (canceled)
